(19) United States
(12) Patent Application Publication
(10) Pub. No.: US 2022/0015840 A1
(43) Pub. Date: Jan. 20, 2022

(54) MODULAR ROBOTIC SURGICAL SYSTEMS

(71) Applicant: Auris Health, Inc., Redwood City, CA (US)

(72) Inventors: Charles J. SCHEIB, Loveland, OH (US); Hannah M. J. MCEWEN, Boston, MA (US)

(73) Assignee: Auris Health, Inc., Redwood City, CA (US)

(21) Appl. No.: 16/931,554

(22) Filed: Jul. 17, 2020

(51) Int. Cl. A61B 34/30; A61B 50/13; A61G 13/10; B25J 9/16; B25J 9/06; B25J 5/00

(52) U.S. Cl. CPC A61B 34/30; A61B 50/13; A61G 13/101; A61B 1/307; B25J 9/06; B25J 5/007; A61B 2034/301; B25J 9/1689

(57) ABSTRACT

A robotic surgical system includes a stationary instrument driver comprising a first instrument driver body attached to a distal joint of a first robotic arm and operable to drive one or more functions of a first surgical tool, and a mobile instrument driver comprising an instrument driver carriage translatable along a longitudinal base and operable to drive one or more functions of a second surgical tool, where the longitudinal base is removably attached to a distal joint of a second robotic arm.

### TECHNICAL FIELD

The systems and methods disclosed herein are directed to robotic surgical systems and, more particularly to, systems designed for modular instrument drivers of varying architecture.

### BACKGROUND

Minimally invasive surgical (MIS) instruments are often preferred over traditional open surgical devices due to the reduced post-operative recovery time and minimal scarring. The most common MIS procedure may be endoscopy, and the most common form of endoscopy is laparoscopy, in which one or more small incisions are formed in the abdomen of a patient and a trocar is inserted through the incision to form a pathway that provides access to the abdominal cavity. The cannula and sealing system of the trocar is used to introduce various instruments and tools into the abdominal cavity, as well as to provide insufflation to elevate the abdominal wall above the organs. The instruments can be used to engage and/or treat tissue in a number of ways to achieve a diagnostic or therapeutic effect.

Each surgical tool typically includes an end effector arranged at its distal end. Example end effectors include clamps, graspers, scissors, staplers, suction irrigators, blades (i.e., RF) and needle holders, and are similar to those used in conventional (open) surgery except that the end effector of each tool is separated from its handle by an approximately 12-inch long shaft. A camera or image capture device, such as an endoscope, is also commonly introduced into the abdominal cavity to enable the surgeon to view the surgical field and the operation of the end effectors during operation. The surgeon is able to view the procedure in real-time by means of a visual display in communication with the image capture device.

Various robotic systems have recently been developed to assist in MIS procedures. Robotic systems can allow for more intuitive hand movements by maintaining natural eye-hand axis. Robotic systems can also allow for more degrees of freedom in movement by including a “wrist” joint that creates a more natural hand-like articulation and allows for access to hard to reach spaces. The instrument's end effector can be articulated (moved) using motors and actuators forming part of a computerized motion system. A user (e.g., a surgeon) is able to remotely operate an instrument's end effector by grasping and manipulating in space one or more controllers that communicate with an instrument driver coupled to the surgical instrument. User inputs are processed by a computer system incorporated into the robotic surgical system and the instrument driver responds by actuating the motors and actuators of the motion system. Moving the drive cables and/or other mechanical mechanisms to manipulate the end effector to desired positions and configurations.

Improvements to robotically-enabled medical systems will provide physicians with the ability to perform endoscopic and laparoscopic procedures more effectively and with improved ease.

### SUMMARY OF DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

Embodiments disclosed herein include a robotic surgical system that includes a stationary instrument driver having a first instrument driver body attached to a distal joint of a first robotic arm that operable to drive one or more functions of a first surgical tool and a mobile instrument driver having an instrument driver carriage translatable along a longitudinal base and operable to drive one or more functions of a second surgical tool, wherein the longitudinal base is attached to a distal joint of a second robotic arm. In a further embodiment, the stationary instrument driver and the longitudinal base of the mobile instrument driver are pivotally coupled to the corresponding distal joints. In another further embodiment, the stationary instrument driver is operable to translate a shaft of the first surgical tool, through the stationary instrument drivers. In another further embodiment, the robotic surgical system further includes an instrument driver interface located on at least one distal joint, the instrument driver interface configured to receive the stationary instrument driver and the mobile instrument driver. In another further embodiment, each robotic arm includes at least two elongated linkages pivotally connected at a joint. In another further embodiment, the first robotic arm has at least six degrees of freedom. In another further embodiment the system includes a modular base, wherein a proximal end of each robotic arm and a coupled instrument driver are removably attachable to the surgical system at the modular base. In another further embodiment, the system includes a cart, wherein each robotic arm is removably attachable to an arm mount movably attached to the cart. In another further embodiment, the system includes a table and at least one rail located on a side of the table, wherein each robotic arm is removably attachable to the at least one rail.

Embodiments disclosed herein may further include a robotic surgical system that includes a first type robotic arm having a stationary instrument driver coupled to a distal end of each first type robotic arm and a second type robotic arm having a mobile instrument driver including an instrument driver carriage capable of translation on an elongated base coupled the second distal end of the second type robotic arm. The system includes a modular base located on a proximal end of each of the first and second type robotic arms and operable to removably attach the first and second type robotic arms to the robotic surgical system. In a further embodiment, the system includes at least one elongated rail providing at least one rail interface operable to removably receive the modular base of each of the first and second type robotic arms. In another further embodiment, at least one of the first and second type robotic arms mounted to the at least one rail is movable along a long axis of the at least one rail. In another further embodiment, the stationary instrument driver operates to translate a shaft of a coupled surgical tool through the stationary instrument driver. In another further embodiment, the system includes a modular joint located on the distal end each of the first and second type robotic arms and operable to removably attach one of the stationary instrument driver and the mobile instrument driver.

Embodiments disclosed herein may further include a robotic surgical system that includes at least two robotic arms, each having a proximal end and a distal end. The system includes one or more stationary instrument drivers configured to drive functions of a coupled first surgical tool and one or more mobile instrument driver including an instrument driver carriage capable of translation on a longitudinal base. The one or more stationary instrument drivers and the one or more mobile instrument driver are each removably attached to the robotic system at one of a modular joint located on the distal end of a robotic arm and a modular base located on the proximal end of a robotic arm. In a further embodiment, the stationary instrument driver allows translation of a shaft of a coupled first surgical tool through the stationary instrument driver. In another further embodiment, the system includes a table and at least one rail located on a side of the table, wherein the at least two robotic arms attach to the at least one rail. In another further embodiment, the system includes a cart, wherein the at least two robotic arms removably attach to an arm mount movably attached to the cart. In another further embodiment, each of at least two robotic arms comprise at least two elongated linkages pivotally connected at joints.

Embodiments disclosed herein may further include a method that includes attaching a stationary instrument driver to a distal joint of a first robotic arm of a first type and attaching a mobile instrument driver to a distal joint of a second robotic arm of a second type, the mobile instrument driver including an instrument driver carriage translatable along an elongated base. The method also includes driving one or more functions of a first surgical tool with the stationary instrument driver and driving one or more functions of a second surgical tool with the mobile instrument driver.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3A illustrates an embodiment of the robotic system of FIG. 1 arranged for ureteroscopy.

FIG. 3B illustrates an embodiment of the robotic system of FIG. 1 arranged for a vascular procedure.

FIG. 4 illustrates an embodiment of a table-based robotic system arranged for a bronchoscopy procedure.

FIG. 5 provides an alternative view of the robotic system of FIG. 4.

FIG. 6 illustrates an example system configured to stow robotic arm(s).

FIG. 7A illustrates an embodiment of a table-based robotic system configured for an ureteroscopy procedure.

FIG. 7B illustrates an embodiment of a table-based robotic system configured for a laparoscopic procedure.

FIG. 7C illustrates an embodiment of the table-based robotic system of FIGS. 4-7B with pitch or tilt adjustment.

FIG. 8 provides a detailed illustration of the interface between the table and the column of the table-based robotic system of FIGS. 4-7.

FIG. 9A illustrates an alternative embodiment of a table-based robotic system.

FIG. 9B illustrates an end view of the table-based robotic system of FIG. 9A.

FIG. 9C illustrates an end view of a table-based robotic system with robotic arms attached thereto.

FIG. 10 illustrates an exemplary instrument driver.

FIG. 11 illustrates an exemplary medical instrument with a paired instrument driver.

FIG. 12 illustrates an alternative design for an instrument driver and instrument where the axes of the drive units are parallel to the axis of the elongated shaft of the instrument.

FIG. 13 illustrates an instrument having an instrument-based insertion architecture.

FIG. 14 illustrates an exemplary controller.

FIG. 15 depicts a block diagram illustrating a localization system that estimates a location of one or more elements of the robotic systems of FIGS. 1-7C, such as the location of the instrument of FIGS. 11-13, in accordance to an example embodiment.

FIG. 16 is an isometric side view of an example surgical tool that may incorporate some or all of the principles of the present disclosure.

FIG. 17A is an isometric view of the surgical tool of FIG. 16 releasably coupled to an example instrument driver, according to one or more embodiments.

FIG. 17B provides separated isometric end views of the instrument driver of FIG. 17A and the surgical tool of FIG. 16.

FIG. 18 illustrates an end view of a table-based robotic system with robotic arms including modular joints attached thereto.

FIG. 19A is a perspective view of a stationary instrument driver of a robotic arm with the surgical tool in a proximal orientation.

FIG. 19B is a perspective view of a stationary instrument driver of a robotic arm with the surgical tool in a distal orientation.

FIG. 19C is a perspective view of a mobile instrument driver of a robotic arm with the surgical tool in a proximal orientation.

FIG. 19D is a perspective view of a mobile instrument driver of a robotic arm with the surgical tool in a distal orientation.

FIG. 20 illustrates an end view of a table-based robotic system with robotic arms including modular bases attached thereto.

FIG. 21 is a perspective view of a table-based robotic system with robotic arms including both stationary instrument drivers and mobile instrument drivers.

### DETAILED DESCRIPTION

### 1. Overview

Aspects of the present disclosure may be integrated into a robotically-enabled medical system capable of performing a variety of medical procedures, including both minimally invasive (e.g., laparoscopy) and non-invasive (e.g., endoscopy) procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

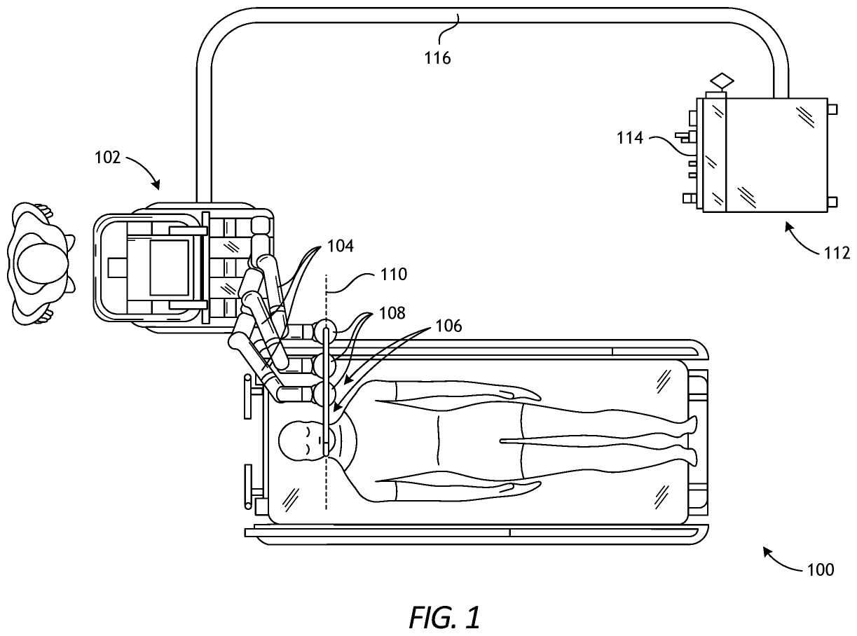

The robotically-enabled medical system may be configured in a variety of ways depending on the particular procedure. FIG. 1 illustrates an embodiment of a cart-based robotically-enabled system 100 arranged for a diagnostic and/or therapeutic bronchoscopy procedure. For a bronchoscopy procedure, the robotic system 100 may include a cart 102 having one or more robotic arms 104 (three shown) to deliver a medical instrument (alternately referred to as a “surgical tool”), such as a steerable endoscope 106 (e.g., a procedure-specific bronchoscope for bronchoscopy), to a natural orifice access point (i.e., the mouth of the patient) to deliver diagnostic and/or therapeutic tools. As shown, the cart 102 may be positioned proximate to the patient's upper torso in order to provide access to the access point. Similarly, the robotic arms 104 may be actuated to position the bronchoscope relative to the access point. The arrangement in FIG. 1 may also be utilized when performing a gastro-intestinal (GI) procedure with a gastroscope, a specialized endoscope for GI procedures.

Once the cart 102 is properly positioned adjacent the patient, the robotic arms 104 are operated to insert the steerable endoscope 106 into the patient robotically, manually, or a combination thereof. The steerable endoscope 106 may comprise at least two telescoping parts, such as an inner leader portion and an outer sheath portion, where each portion is coupled to a separate instrument driver of a set of instrument drivers 108. As illustrated, each instrument driver 108 is coupled to the distal end of a corresponding one of the robotic arms 104. This linear arrangement of the instrument drivers 108, which facilitates coaxially aligning the leader portion with the sheath portion, creates a “virtual rail” 110 that may be repositioned in space by manipulating the robotic arms 104 into different angles and/or positions. Translation of the instrument drivers 108 along the virtual rail 110 telescopes the inner leader portion relative to the outer sheath portion, thus effectively advancing or retracting the endoscope 106 relative to the patient.

As illustrated, the virtual rail 110 (and other virtual rails described herein) is depicted in the drawings using dashed lines, thus not constituting any physical structure of the system 100. The angle of the virtual rail 110 may be adjusted, translated, and pivoted based on clinical application or physician preference. For example, in bronchoscopy, the angle and position of the virtual rail 110 as shown represents a compromise between providing physician access to the endoscope 106 while minimizing friction that results from bending the endoscope 106 into the patient's mouth.

After insertion into the patient's mouth, the endoscope 106 may be directed down the patient's trachea and lungs using precise commands from the robotic system 100 until reaching a target destination or operative site. In order to enhance navigation through the patient's lung network and/or reach the desired target, the endoscope 106 may be manipulated to telescopically extend the inner leader portion from the outer sheath portion to obtain enhanced articulation and greater bend radius. The use of separate instrument drivers 108 also allows the leader portion and sheath portion to be driven independent of each other.

For example, the endoscope 106 may be directed to deliver a biopsy needle to a target, such as, for example, a lesion or nodule within the lungs of a patient. The needle may be deployed down a working channel that runs the length of the endoscope 106 to obtain a tissue sample to be analyzed by a pathologist. Depending on the pathology results, additional tools may be deployed down the working channel of the endoscope for additional biopsies. After identifying a tissue sample to be malignant, the endoscope 106 may endoscopically deliver tools to resect the potentially cancerous tissue. In some instances, diagnostic and therapeutic treatments can be delivered in separate procedures. In those circumstances, the endoscope 106 may also be used to deliver a fiducial marker to “mark” the location of a target nodule as well. In other instances, diagnostic and therapeutic treatments may be delivered during the same procedure.

The system 100 may also include a movable tower 112, which may be connected via support cables to the cart 102 to provide support for controls, electronics, fluidics, optics, sensors, and/or power to the cart 102. Placing such functionality in the tower 112 allows for a smaller form factor cart 102 that may be more easily adjusted and/or re-positioned by an operating physician and his/her staff. Additionally, the division of functionality between the cart/table and the support tower 112 reduces operating room clutter and facilitates improving clinical workflow. While the cart 102 may be positioned close to the patient, the tower 112 may alternatively be stowed in a remote location to stay out of the way during a procedure.

In support of the robotic systems described above, the tower 112 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 112 or the cart 102, may control the entire system or sub-system(s) thereof. For example, when executed by a processor of the computer system, the instructions may cause the components of the robotic system to actuate the relevant carriages and arm mounts, actuate the robotics arms, and control the medical instruments. For example, in response to receiving the control signal, motors in the joints of the robotic arms 104 may position the arms into a certain posture or angular orientation.

The tower 112 may also include one or more of a pump, flow meter, valve control, and/or fluid access in order to provide controlled irrigation and aspiration capabilities to the system 100 that may be deployed through the endoscope 106. These components may also be controlled using the computer system of the tower 112. In some embodiments, irrigation and aspiration capabilities may be delivered directly to the endoscope 106 through separate cable(s).

The tower 112 may include a voltage and surge protector designed to provide filtered and protected electrical power to the cart 102, thereby avoiding placement of a power transformer and other auxiliary power components in the cart 102, resulting in a smaller, more moveable cart 102.

The tower 112 may also include support equipment for sensors deployed throughout the robotic system 100. For example, the tower 112 may include opto-electronics equipment for detecting, receiving, and processing data received from optical sensors or cameras throughout the robotic system 100. In combination with the control system, such opto-electronics equipment may be used to generate real-time images for display in any number of consoles deployed throughout the system, including in the tower 112. Similarly, the tower 112 may also include an electronic subsystem for receiving and processing signals received from deployed electromagnetic (EM) sensors. The tower 112 may also be used to house and position an EM field generator for detection by EM sensors in or on the medical instrument.

The tower 112 may also include a console 114 in addition to other consoles available in the rest of the system, e.g., a console mounted to the cart 102. The console 114 may include a user interface and a display screen (e.g., a touchscreen) for the physician operator. Consoles in the system 100 are generally designed to provide both robotic controls as well as pre-operative and real-time information of the procedure, such as navigational and localization information of the endoscope 106. When the console 114 is not the only console available to the physician, it may be used by a second operator, such as a nurse, to monitor the health or vitals of the patient and the operation of system, as well as provide procedure-specific data, such as navigational and localization information. In other embodiments, the console 114 may be housed in a body separate from the tower 112.

The tower 112 may be coupled to the cart 102 and endoscope 106 through one or more cables 116 connections. In some embodiments, support functionality from the tower 112 may be provided through a single cable 116 extending to the cart 102, thus simplifying and de-cluttering the operating room. In other embodiments, specific functionality may be coupled in separate cabling and connections. For example, while power may be provided through a single power cable to the cart 102, support for controls, optics, fluidics, and/or navigation may be provided through one or more separate cables.

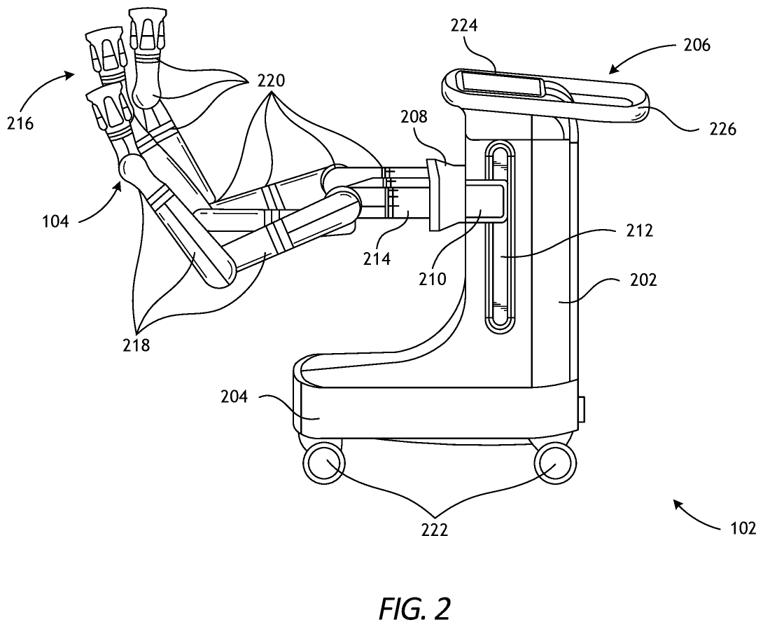

FIG. 2 provides a detailed illustration of an embodiment of the cart 102 from the cart-based robotically-enabled system 100 of FIG. 1. The cart 102 generally includes an elongated support structure 202 (also referred to as a “column”), a cart base 204, and a console 206 at the top of the column 202. The column 202 may include one or more carriages, such as a carriage 208 (alternatively “arm support”) for supporting the deployment of the robotic arms 104. The carriage 208 may include individually configurable arm mounts that rotate along a perpendicular axis to adjust the base 214 of the robotic arms 104 for better positioning relative to the patient. The carriage 208 also includes a carriage interface 210 that allows the carriage 208 to vertically translate along the column 202.

The carriage interface 210 is connected to the column 202 through slots, such as slot 212, that are positioned on opposite sides of the column 202 to guide the vertical translation of the carriage 208. The slot 212 contains a vertical translation interface to position and hold the carriage 208 at various vertical heights relative to the cart base 204. Vertical translation of the carriage 208 allows the cart 102 to adjust the reach of the robotic arms 104 to meet a variety of table heights, patient sizes, and physician preferences. Similarly, the individually configurable arm mounts on the carriage 208 allow a base 214 of the robotic arms 104 to be angled in a variety of configurations.

In some embodiments, the slot 212 may be supplemented with slot covers (not shown) that are flush and parallel to the slot surface to prevent dirt and fluid ingress into the internal chambers of the column 202 and the vertical translation interface as the carriage 208 vertically translates. The slot covers may be deployed through pairs of spring spools positioned near the vertical top and bottom of the slot 212. The covers are coiled within the spools until deployed to extend and retract from their coiled state as the carriage 208 vertically translates up and down. The spring-loading of the spools provides force to retract the cover into a spool when carriage 208 translates towards the spool, while also maintaining a tight seal when the carriage 208 translates away from the spool. The covers may be connected to the carriage 208 using, for example, brackets in the carriage interface 210 to ensure proper extension and retraction of the cover as the carriage 208 translates.

The column 202 may internally comprise mechanisms, such as gears and motors, that are designed to use a vertically aligned lead screw to translate the carriage 208 in a mechanized fashion in response to control signals generated in response to user inputs, e.g., inputs from the console 206.

The robotic arms 104 may generally comprise robotic arm bases 214 and end effectors 216 (three shown), separated by a series of linkages 218 connected by a corresponding series of joints 220, each joint 220 including an independent actuator, and each actuator including an independently controllable motor. Each independently controllable joint 220 represents an independent degree of freedom available to the corresponding robotic arm 104. In the illustrated embodiment, each arm 104 has seven joints 220, thus providing seven degrees of freedom. A multitude of joints 220 result in a multitude of degrees of freedom, allowing for “redundant” degrees of freedom. Redundant degrees of freedom allow the robotic arms 104 to position their respective end effectors 216 at a specific position, orientation, and trajectory in space using different linkage positions and joint angles. This allows for the system 100 to position and direct a medical instrument from a desired point in space while allowing the physician to move the arm joints 220 into a clinically advantageous position away from the patient to create greater access, while avoiding arm collisions.

The cart base 204 balances the weight of the column 202, carriage 208, and arms 104 over the floor. Accordingly, the cart base 204 houses heavier components, such as electronics, motors, power supply, as well as components that either enable movement and/or immobilize the cart. For example, the cart base 204 includes rolling casters 222 that allow for the cart to easily move around the room prior to a procedure. After reaching an appropriate position, the casters 222 may be immobilized using wheel locks to hold the cart 102 in place during the procedure.

Positioned at the vertical end of the column 202, the console 206 allows for both a user interface for receiving user input and a display screen (or a dual-purpose device such as, for example, a touchscreen 224) to provide the physician user with both pre-operative and intra-operative data. Potential pre-operative data on the touchscreen 224 may include pre-operative plans, navigation and mapping data derived from pre-operative computerized tomography (CT) scans, and/or notes from pre-operative patient interviews. Intra-operative data on the touchscreen 224 may include optical information provided from the tool, sensor and coordinate information from sensors, as well as vital patient statistics, such as respiration, heart rate, and/or pulse. The console 206 may be positioned and tilted to allow a physician to access the console from the side of the column 202 opposite carriage 208. From this position, the physician may view the console 206, the robotic arms 104, and the patient while operating the console 206 from behind the cart 102. As shown, the console 206 also includes a handle 226 to assist with maneuvering and stabilizing cart 102.

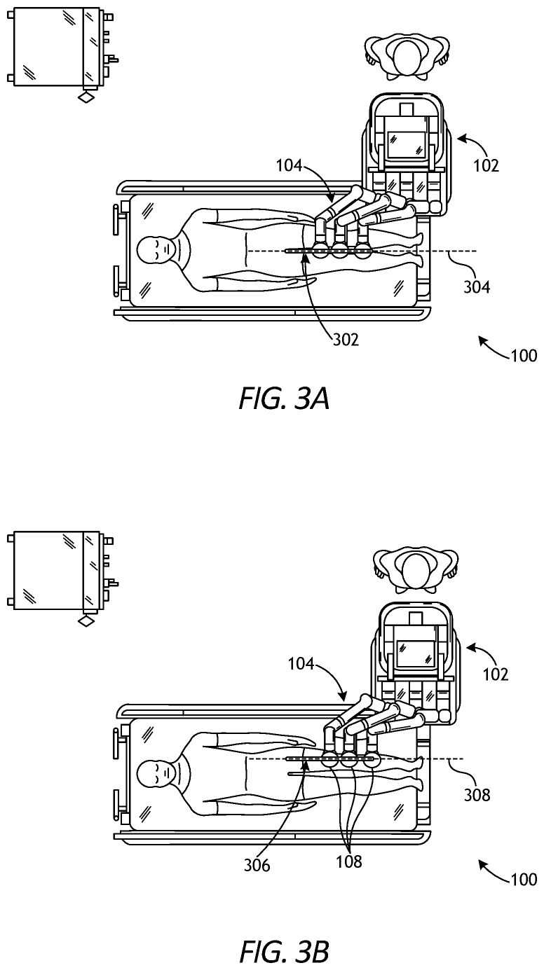

FIG. 3A illustrates an embodiment of the system 100 of FIG. 1 arranged for ureteroscopy. In a ureteroscopic procedure, the cart 102 may be positioned to deliver a ureteroscope 302, a procedure-specific endoscope designed to traverse a patient's urethra and ureter, to the lower abdominal area of the patient. In ureteroscopy, it may be desirable for the ureteroscope 302 to be directly aligned with the patient's urethra to reduce friction and forces on the sensitive anatomy. As shown, the cart 102 may be aligned at the foot of the table to allow the robotic arms 104 to position the ureteroscope 302 for direct linear access to the patient's urethra. From the foot of the table, the robotic arms 104 may insert the ureteroscope 302 along a virtual rail 304 directly into the patient's lower abdomen through the urethra.

After insertion into the urethra, using similar control techniques as in bronchoscopy, the ureteroscope 302 may be navigated into the bladder, ureters, and/or kidneys for diagnostic and/or therapeutic applications. For example, the ureteroscope 302 may be directed into the ureter and kidneys to break up kidney stone build-up using a laser or ultrasonic lithotripsy device deployed down a working channel of the ureteroscope 302. After lithotripsy is complete, the resulting stone fragments may be removed using baskets deployed down the working channel of the ureteroscope 302.

FIG. 3B illustrates another embodiment of the system 100 of FIG. 1 arranged for a vascular procedure. In a vascular procedure, the system 100 may be configured such that the cart 102 may deliver a medical instrument 306, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 102 may be positioned towards the patient's legs and lower abdomen to allow the robotic arms 104 to provide a virtual rail 308 with direct linear access to the femoral artery access point in the patient's thigh/hip region. After insertion into the artery, the medical instrument 306 may be directed and advanced by translating the instrument drivers 108. Alternatively, the cart 102 may be positioned around the patient's upper abdomen in order to reach alternative vascular access points, such as, for example, the carotid and brachial arteries near the patient's shoulder and wrist.

### B. Robotic System—Table.

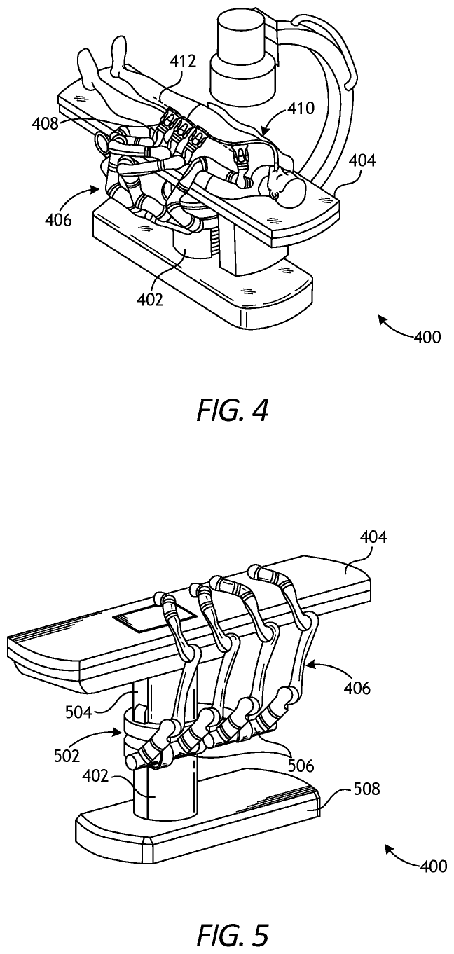

Embodiments of the robotically-enabled medical system may also incorporate the patient's table. Incorporation of the table reduces the amount of capital equipment within the operating room by removing the cart, which allows greater access to the patient. FIG. 4 illustrates an embodiment of such a robotically-enabled system 400 arranged for a bronchoscopy procedure. As illustrated, the system 400 includes a support structure or column 402 for supporting platform 404 (shown as a “table” or “bed”) over the floor. Much like in the cart-based systems, the end effectors of the robotic arms 406 of the system 400 comprise instrument drivers 408 that are designed to manipulate an elongated medical instrument, such as a bronchoscope 410, through or along a virtual rail 412 formed from the linear alignment of the instrument drivers 408. In practice, a C-arm for providing fluoroscopic imaging may be positioned over the patient's upper abdominal area by placing the emitter and detector around the table 404.

FIG. 5 provides an alternative view of the system 400 without the patient and medical instrument for discussion purposes. As shown, the column 402 may include one or more carriages 502 shown as ring-shaped in the system 400, from which the one or more robotic arms 406 may be based. The carriages 502 may translate along a vertical column interface 504 that runs the length (height) of the column 402 to provide different vantage points from which the robotic arms 406 may be positioned to reach the patient. The carriage(s) 502 may rotate around the column 402 using a mechanical motor positioned within the column 402 to allow the robotic arms 406 to have access to multiples sides of the table 404, such as, for example, both sides of the patient. In embodiments with multiple carriages 502, the carriages 502 may be individually positioned on the column 402 and may translate and/or rotate independent of the other carriages 502. While carriages 502 need not surround the column 402 or even be circular, the ring-shape as shown facilitates rotation of the carriages 502 around the column 402 while maintaining structural balance. Rotation and translation of the carriages 502 allows the system 400 to align medical instruments, such as endoscopes and laparoscopes, into different access points on the patient.

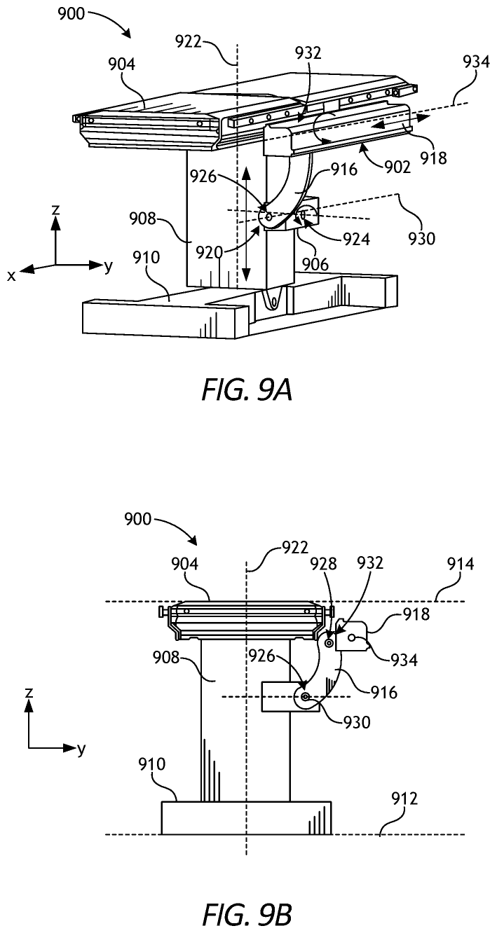

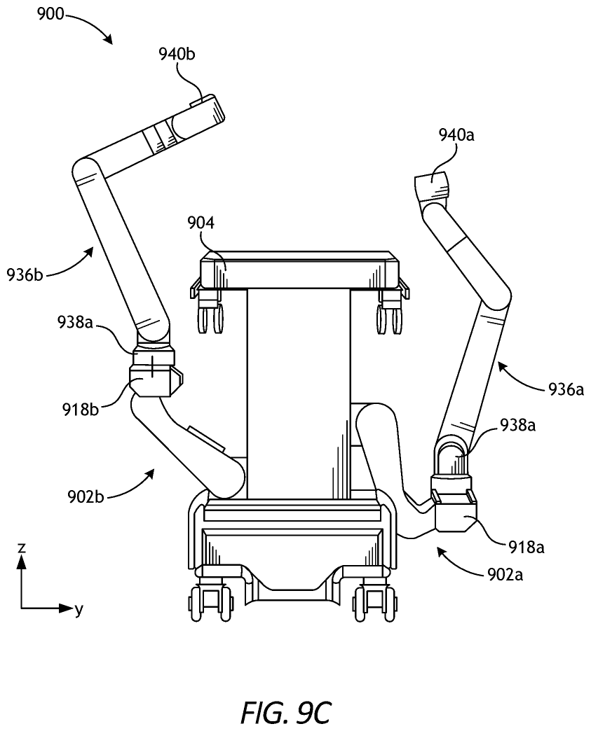

In other embodiments (discussed in greater detail below with respect to FIG. 9A), the system 400 can include a patient table or bed with adjustable arm supports in the form of bars or rails extending alongside it. One or more robotic arms 406 (e.g., via a shoulder with an elbow j oint) can be attached to the adjustable arm supports, which can be vertically adjusted. By providing vertical adjustment, the robotic arms 406 are advantageously capable of being stowed compactly beneath the patient table or bed, and subsequently raised during a procedure.

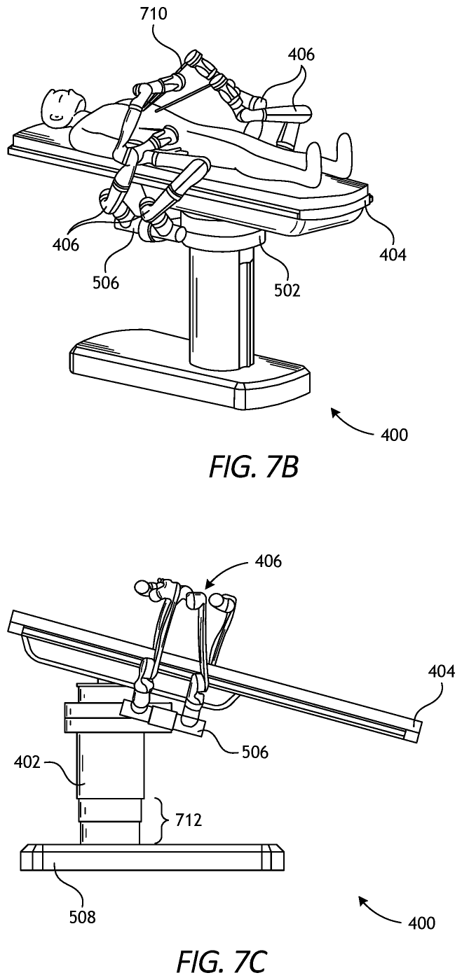

The arms 406 may be mounted on the carriages 502 through a set of arm mounts 506 comprising a series of joints that may individually rotate and/or telescopically extend to provide additional configurability to the robotic arms 406. Additionally, the arm mounts 506 may be positioned on the carriages 502 such that when the carriages 502 are appropriately rotated, the arm mounts 506 may be positioned on either the same side of the table 404 (as shown in FIG. 5), on opposite sides of table 404 (as shown in FIG. 7B), or on adjacent sides of the table 404 (not shown).

The column 402 structurally provides support for the table 404, and a path for vertical translation of the carriages 502. Internally, the column 402 may be equipped with lead screws for guiding vertical translation of the carriages, and motors to mechanize the translation of said carriages based the lead screws. The column 402 may also convey power and control signals to the carriage 502 and robotic arms 406 mounted thereon.

A table base 508 serves a similar function as the cart base 204 of the cart 102 shown in FIG. 2, housing heavier components to balance the table/bed 404, the column 402, the carriages 502, and the robotic arms 406. The table base 508 may also incorporate rigid casters to provide stability during procedures. Deployed from the bottom of the table base 508, the casters may extend in opposite directions on both sides of the base 508 and retract when the system 400 needs to be moved.

In some embodiments, the system 400 may also include a tower (not shown) that divides the functionality of system 400 between table and tower to reduce the form factor and bulk of the table 404. As in earlier disclosed embodiments, the tower may provide a variety of support functionalities to the table 404, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 508 for potential stowage of the robotic arms 406. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

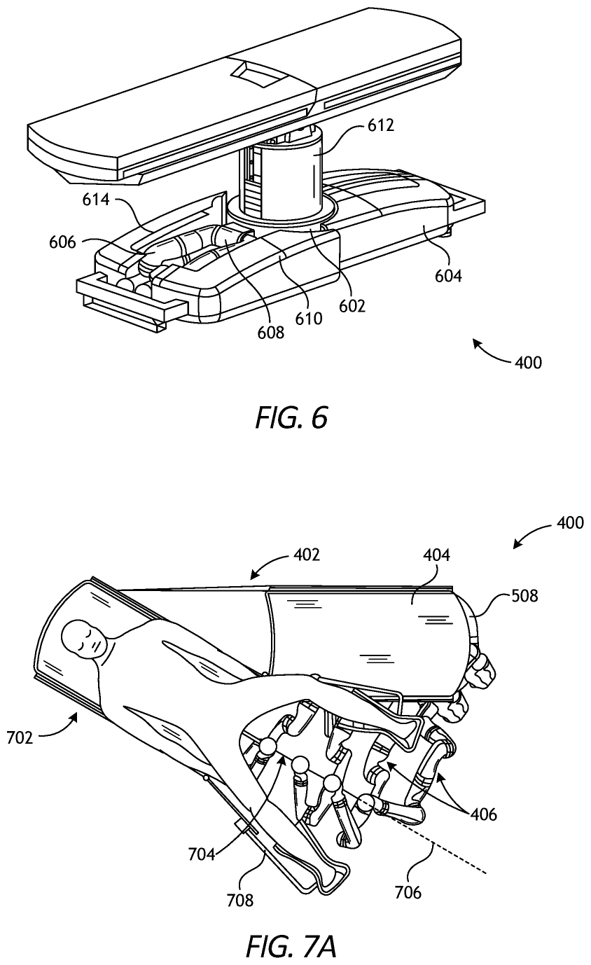

In some embodiments, a table base may stow and store the robotic arms when not in use. FIG. 6 illustrates an embodiment of the system 400 that is configured to stow robotic arms in an embodiment of the table-based system. In the system 400, one or more carriages 602 (one shown) may be vertically translated into a base 604 to stow one or more robotic arms 606, one or more arm mounts 608, and the carriages 602 within the base 604. Base covers 610 may be translated and retracted open to deploy the carriages 602, the arm mounts 608, and the arms 606 around the column 612, and closed to stow and protect them when not in use. The base covers 610 may be sealed with a membrane 614 along the edges of its opening to prevent dirt and fluid ingress when closed.

FIG. 7A illustrates an embodiment of the robotically-enabled table-based system 400 configured for a ureteroscopy procedure. In ureteroscopy, the table 404 may include a swivel portion 702 for positioning a patient off-angle from the column 402 and the table base 508. The swivel portion 702 may rotate or pivot around a pivot point (e.g., located below the patient's head) in order to position the bottom portion of the swivel portion 702 away from the column 402. For example, the pivoting of the swivel portion 702 allows a C-arm (not shown) to be positioned over the patient's lower abdomen without competing for space with the column (not shown) below table 404. By rotating the carriage (not shown) around the column 402, the robotic arms 406 may directly insert a ureteroscope 704 along a virtual rail 706 into the patient's groin area to reach the urethra. In ureteroscopy, stirrups 708 may also be fixed to the swivel portion 702 of the table 404 to support the position of the patient's legs during the procedure and allow clear access to the patient's groin area.

FIG. 7B illustrates an embodiment of the system 400 configured for a laparoscopic procedure. In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. As shown in FIG. 7B, the carriages 502 of the system 400 may be rotated and vertically adjusted to position pairs of the robotic arms 406 on opposite sides of the table 404, such that an instrument 710 may be positioned using the arm mounts 506 to be passed through minimal incisions on both sides of the patient to reach his/her abdominal cavity.

To accommodate laparoscopic procedures, the system 400 may also tilt the platform to a desired angle. FIG. 7C illustrates an embodiment of the system 400 with pitch or tilt adjustment. As shown in FIG. 7C, the system 400 may accommodate tilt of the table 404 to position one portion of the table 404 at a greater distance from the floor than the other. Additionally, the arm mounts 506 may rotate to match the tilt such that the arms 406 maintain the same planar relationship with table 404. To accommodate steeper angles, the column 402 may also include telescoping portions 712 that allow vertical extension of the column 402 to keep the table 404 from touching the floor or colliding with the base 508.

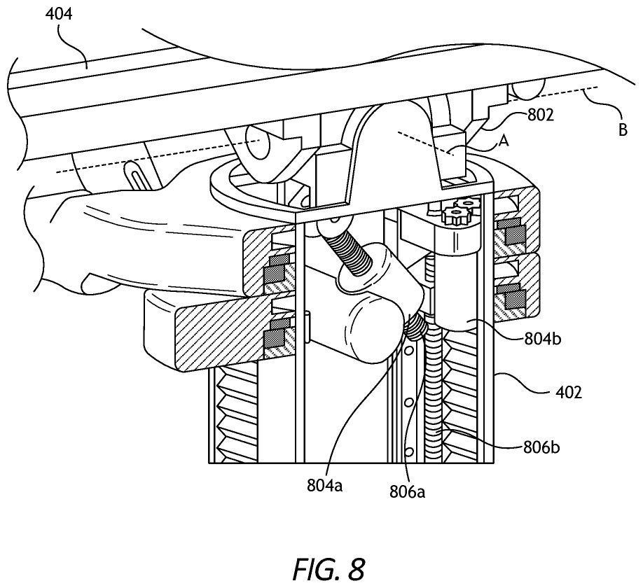

FIG. 8 provides a detailed illustration of the interface between the table 404 and the column 402. Pitch rotation mechanism 802 may be configured to alter the pitch angle of the table 404 relative to the column 402 in multiple degrees of freedom. The pitch rotation mechanism 802 may be enabled by the positioning of orthogonal axes A and B at the column-table interface, each axis actuated by a separate motor 804a and 804b responsive to an electrical pitch angle command. Rotation along one screw 806a would enable tilt adjustments in one axis A, while rotation along another screw 806b would enable tilt adjustments along the other axis B. In some embodiments, a ball joint can be used to alter the pitch angle of the table 404 relative to the column 402 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

FIGS. 9A and 9B illustrate isometric and end views, respectively, of an alternative embodiment of a table-based surgical robotics system 900. The surgical robotics system 900 includes one or more adjustable arm supports 902 that can be configured to support one or more robotic arms (see, for example, FIG. 9C) relative to a table 904. In the illustrated embodiment, a single adjustable arm support 902 is shown, though an additional arm support can be provided on an opposite side of the table 904. The adjustable arm support 902 can be configured so that it can move relative to the table 904 to adjust and/or vary the position of the adjustable arm support 902 and/or any robotic arms mounted thereto relative to the table 904. For example, the adjustable arm support 902 may be adjusted in one or more degrees of freedom relative to the table 904. The adjustable arm support 902 provides high versatility to the system 900, including the ability to easily stow the one or more adjustable arm supports 902 and any robotics arms attached thereto beneath the table 904. The adjustable arm support 902 can be elevated from the stowed position to a position below an upper surface of the table 904. In other embodiments, the adjustable arm support 902 can be elevated from the stowed position to a position above an upper surface of the table 904.

The adjustable arm support 902 can provide several degrees of freedom, including lift, lateral translation, tilt, etc. In the illustrated embodiment of FIGS. 9A and 9B, the arm support 902 is configured with four degrees of freedom, which are illustrated with arrows in FIG. 9A. A first degree of freedom allows for adjustment of the adjustable arm support 902 in the z-direction (“Z-lift”). For example, the adjustable arm support 902 can include a carriage 906 configured to move up or down along or relative to a column 908 supporting the table 904. A second degree of freedom can allow the adjustable arm support 902 to tilt. For example, the adjustable arm support 902 can include a rotary joint, which can allow the adjustable arm support 902 to be aligned with the bed in a Trendelenburg position. A third degree of freedom can allow the adjustable arm support 902 to “pivot up,” which can be used to adjust a distance between a side of the table 904 and the adjustable arm support 902. A fourth degree of freedom can permit translation of the adjustable arm support 902 along a longitudinal length of the table.

The surgical robotics system 900 in FIGS. 9A and 9B can comprise a table 904 supported by a column 908 that is mounted to a base 910. The base 910 and the column 908 support the table 904 relative to a support surface. A floor axis 912 and a support axis 914 are shown in FIG. 9B.

The adjustable arm support 902 can be mounted to the column 908. In other embodiments, the arm support 902 can be mounted to the table 904 or the base 910. The adjustable arm support 902 can include a carriage 906, a bar or rail connector 916 and a bar or rail 918. In some embodiments, one or more robotic arms mounted to the rail 918 can translate and move relative to one another.

The carriage 906 can be attached to the column 908 by a first joint 920, which allows the carriage 906 to move relative to the column 908 (e.g., such as up and down a first or vertical axis 922). The first joint 920 can provide the first degree of freedom (“Z-lift”) to the adjustable arm support 902. The adjustable arm support 902 can include a second joint 924, which provides the second degree of freedom (tilt) for the adjustable arm support 902. The adjustable arm support 902 can include a third joint 926, which can provide the third degree of freedom (“pivot up”) for the adjustable arm support 902. An additional joint 928 (shown in FIG. 9B) can be provided that mechanically constrains the third joint 926 to maintain an orientation of the rail 918 as the rail connector 916 is rotated about a third axis 930. The adjustable arm support 902 can include a fourth joint 932, which can provide a fourth degree of freedom (translation) for the adjustable arm support 902 along a fourth axis 934.

FIG. 9C illustrates an end view of the surgical robotics system 900 with two adjustable arm supports 902a and 902b mounted on opposite sides of the table 904. A first robotic arm 936a is attached to the first bar or rail 918a of the first adjustable arm support 902a. The first robotic arm 936a includes a base 938a attached to the first rail 918a. The distal end of the first robotic arm 936a includes an instrument drive mechanism or input 940a that can attach to one or more robotic medical instruments or tools. Similarly, the second robotic arm 936b includes a base 938a attached to the second rail 918b. The distal end of the second robotic arm 936b includes an instrument drive mechanism or input 940b configured to attach to one or more robotic medical instruments or tools.

In some embodiments, one or more of the robotic arms 936a,b comprises an arm with seven or more degrees of freedom. In some embodiments, one or more of the robotic arms 936a,b can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 938a,b (1-degree of freedom including translation). In some embodiments, the insertion degree of freedom can be provided by the robotic arm 936a,b, while in other embodiments, the instrument itself provides insertion via an instrument-based insertion architecture.

### C. Instrument Driver & Interface.

The end effectors of a system's robotic arms comprise (i) an instrument driver (alternatively referred to as “instrument drive mechanism,” “instrument device manipulator,” and “drive input”) that incorporate electro-mechanical means for actuating the medical instrument, and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

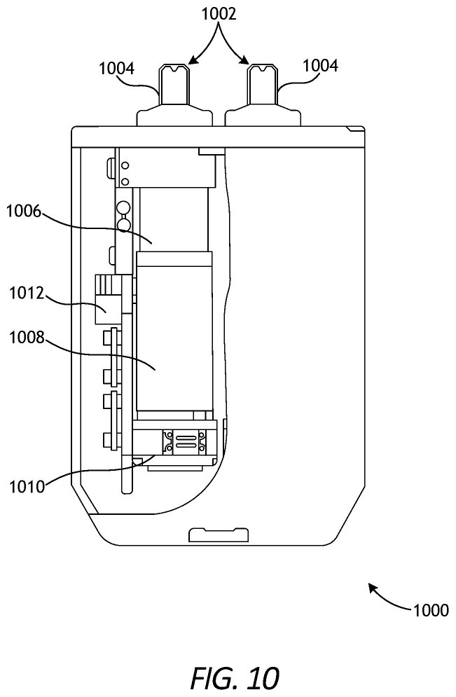

FIG. 10 illustrates an example instrument driver 1000, according to one or more embodiments. Positioned at the distal end of a robotic arm, the instrument driver 1000 comprises of one or more drive outputs 1002 arranged with parallel axes to provide controlled torque to a medical instrument via corresponding drive shafts 1004. Each drive output 1002 comprises an individual drive shaft 1004 for interacting with the instrument, a gear head 1006 for converting the motor shaft rotation to a desired torque, a motor 1008 for generating the drive torque, and an encoder 1010 to measure the speed of the motor shaft and provide feedback to control circuitry 1012, which can also be used for receiving control signals and actuating the drive output 1002. Each drive output 1002 being independent controlled and motorized, the instrument driver 1000 may provide multiple (at least two shown in FIG. 10) independent drive outputs to the medical instrument. In operation, the control circuitry 1012 receives a control signal, transmits a motor signal to the motor 1008, compares the resulting motor speed as measured by the encoder 1010 with the desired speed, and modulates the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

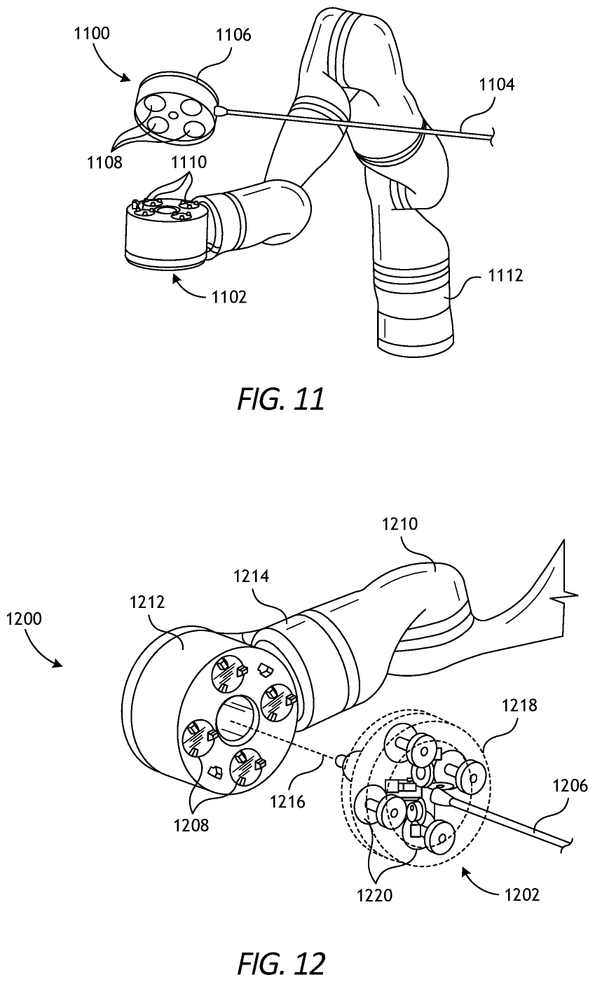

FIG. 11 illustrates an example medical instrument 1100 with a paired instrument driver 1102. Like other instruments designed for use with a robotic system, the medical instrument 1100 (alternately referred to as a “surgical tool”) comprises an elongated shaft 1104 (or elongate body) and an instrument base 1106. The instrument base 1106, also referred to as an “instrument handle” due to its intended design for manual interaction by the physician, may generally comprise rotatable drive inputs 1108, e.g., receptacles, pulleys or spools, that are designed to be mated with drive outputs 1110 that extend through a drive interface on the instrument driver 1102 at the distal end of a robotic arm 1112. When physically connected, latched, and/or coupled, the mated drive inputs 1108 of the instrument base 1106 may share axes of rotation with the drive outputs 1110 in the instrument driver 1102 to allow the transfer of torque from the drive outputs 1110 to the drive inputs 1108. In some embodiments, the drive outputs 1110 may comprise splines that are designed to mate with receptacles on the drive inputs 1108.

The elongated shaft 1104 is designed to be delivered through either an anatomical opening or lumen, e.g., as in endoscopy, or a minimally invasive incision, e.g., as in laparoscopy. The elongated shaft 1104 may be either flexible (e.g., having properties similar to an endoscope) or rigid (e.g., having properties similar to a laparoscope) or contain a customized combination of both flexible and rigid portions. When designed for laparoscopy, the distal end of the shaft 1104 may be connected to an end effector extending from a jointed wrist formed from a clevis with at least one degree of freedom and a surgical tool or medical instrument, such as, for example, a grasper or scissors, that may be actuated based on force from the tendons as the drive inputs 1008 rotate in response to torque received from the drive outputs 1110 of the instrument driver 1102. When designed for endoscopy, the distal end of the flexible elongated shaft 1104 may include a steerable or controllable bending section that may be articulated and bent based on torque received from the drive outputs 1110 of the instrument driver 1102.

In some embodiments, torque from the instrument driver 1102 is transmitted down the elongated shaft 1104 using tendons along the shaft 1104. These individual tendons, such as pull wires, may be individually anchored to individual drive inputs 1108 within the instrument handle 1106. From the handle 1106, the tendons are directed down one or more pull lumens along the elongated shaft 1104 and anchored at the distal portion of the elongated shaft 1104, or in the wrist at the distal portion of the elongated shaft. During a surgical procedure, such as a laparoscopic, endoscopic, or a hybrid procedure, these tendons may be coupled to a distally mounted end effector, such as a wrist, a grasper, or scissors. Under such an arrangement, torque exerted on the drive inputs 1108 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 1104, where tension from the tendon cause the grasper to close.

In endoscopy, the tendons may be coupled to a bending or articulating section positioned along the elongated shaft 1104 (e.g., at the distal end) via adhesive, control ring, or other mechanical fixation. When fixedly attached to the distal end of a bending section, torque exerted on drive inputs 1108 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 1104 to allow for controlled articulation in the desired bending or articulable sections.

In endoscopy, the elongated shaft 1104 houses a number of components to assist with the robotic procedure. The shaft may comprise of a working channel for deploying surgical tools (or medical instruments), irrigation, and/or aspiration to the operative region at the distal end of the shaft 1104. The shaft 1104 may also accommodate wires and/or optical fibers to transfer signals to/from an optical assembly at the distal tip, which may include of an optical camera. The shaft 1104 may also accommodate optical fibers to carry light from proximally-located light sources, such as light emitting diodes, to the distal end of the shaft.

At the distal end of the instrument 1100, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 11, the drive shaft axes, and thus the drive input axes, are orthogonal to the axis of the elongated shaft. This arrangement, however, complicates roll capabilities for the elongated shaft 1104. Rolling the elongated shaft 1104 along its axis while keeping the drive inputs 1108 static results in undesirable tangling of the tendons as they extend off the drive inputs 1108 and enter pull lumens within the elongated shaft 1104. The resulting entanglement of such tendons may disrupt any control algorithms intended to predict movement of the flexible elongated shaft during an endoscopic procedure.

FIG. 12 illustrates an alternative design for a circular instrument driver 1200 and corresponding instrument 1202 (alternately referred to as a “surgical tool”) where the axes of the drive units are parallel to the axis of the elongated shaft 1206 of the instrument 1202. As shown, the instrument driver 1200 comprises four drive units with corresponding drive outputs 1208 aligned in parallel at the end of a robotic arm 1210. The drive units and their respective drive outputs 1208 are housed in a rotational assembly 1212 of the instrument driver 1200 that is driven by one of the drive units within the assembly 1212. In response to torque provided by the rotational drive unit, the rotational assembly 1212 rotates along a circular bearing that connects the rotational assembly 1212 to a non-rotational portion 1214 of the instrument driver 1200. Power and control signals may be communicated from the non-rotational portion 1214 of the instrument driver 1200 to the rotational assembly 1212 through electrical contacts maintained through rotation by a brushed slip ring connection (not shown). In other embodiments, the rotational assembly 1212 may be responsive to a separate drive unit that is integrated into the non-rotatable portion 1214, and thus not in parallel with the other drive units. The rotational assembly 1212 allows the instrument driver 1200 to rotate the drive units and their respective drive outputs 1208 as a single unit around an instrument driver axis 1216.

Like earlier disclosed embodiments, the instrument 1202 may include an elongated shaft 1206 and an instrument base 1218 (shown in phantom) including a plurality of drive inputs 1220 (such as receptacles, pulleys, and spools) that are configured to mate with the drive outputs 1208 of the instrument driver 1200. Unlike prior disclosed embodiments, the instrument shaft 1206 extends from the center of the instrument base 1218 with an axis substantially parallel to the axes of the drive inputs 1220, rather than orthogonal as in the design of FIG. 11.

When coupled to the rotational assembly 1212 of the instrument driver 1200, the medical instrument 1202, comprising instrument base 1218 and instrument shaft 1206, rotates in combination with the rotational assembly 1212 about the instrument driver axis 1216. Since the instrument shaft 1206 is positioned at the center of the instrument base 1218, the instrument shaft 1206 is coaxial with the instrument driver axis 1216 when attached. Thus, rotation of the rotational assembly 1212 causes the instrument shaft 1206 to rotate about its own longitudinal axis. Moreover, as the instrument base 1218 rotates with the instrument shaft 1206, any tendons connected to the drive inputs 1220 in the instrument base 1218 are not tangled during rotation. Accordingly, the parallelism of the axes of the drive outputs 1208, the drive inputs 1220, and the instrument shaft 1206 allows for the shaft rotation without tangling any control tendons.

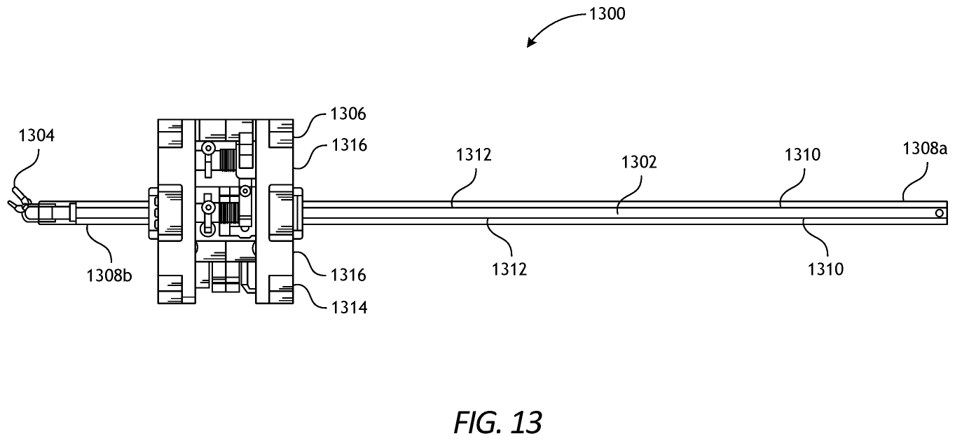

FIG. 13 illustrates a medical instrument 1300 having an instrument based insertion architecture in accordance with some embodiments. The instrument 1300 (alternately referred to as a “surgical tool”) can be coupled to any of the instrument drivers discussed herein above and, as illustrated, can include an elongated shaft 1302, an end effector 1304 connected to the shaft 1302, and a handle 1306 coupled to the shaft 1302. The elongated shaft 1302 comprises a tubular member having a proximal portion 1308a and a distal portion 1308b. The elongated shaft 1302 comprises one or more channels or grooves 1310 along its outer surface and configured to receive one or more wires or cables 1312 therethrough. One or more cables 1312 thus run along an outer surface of the elongated shaft 1302. In other embodiments, the cables 1312 can also run through the elongated shaft 1302. Manipulation of the cables 1312 (e.g., via an instrument driver) results in actuation of the end effector 1304.

The instrument handle 1306, which may also be referred to as an instrument base, may generally comprise an attachment interface 1314 having one or more mechanical inputs 1316, e.g., receptacles, pulleys or spools, that are designed to be reciprocally mated with one or more drive outputs on an attachment surface of an instrument driver.

In some embodiments, the instrument 1300 comprises a series of pulleys or cables that enable the elongated shaft 1302 to translate relative to the handle 1306. In other words, the instrument 1300 itself comprises an instrument-based insertion architecture that accommodates insertion of the instrument, thereby minimizing the reliance on a robot arm to provide insertion of the instrument 1300. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

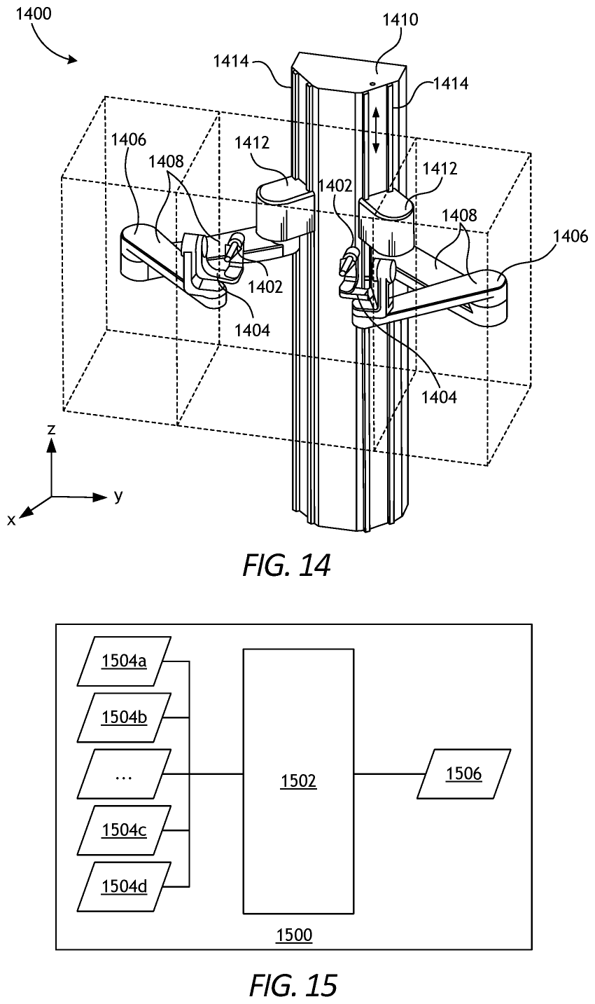

FIG. 14 is a perspective view of an embodiment of a controller 1400. In the present embodiment, the controller 1400 comprises a hybrid controller that can have both impedance and admittance control. In other embodiments, the controller 1400 can utilize just impedance or passive control. In other embodiments, the controller 1400 can utilize just admittance control. By being a hybrid controller, the controller 1400 advantageously can have a lower perceived inertia while in use.

In the illustrated embodiment, the controller 1400 is configured to allow manipulation of two medical instruments, and includes two handles 1402. Each of the handles 1402 is connected to a gimbal 1404, and each gimbal 1404 is connected to a positioning platform 1406.

As shown in FIG. 14, each positioning platform 1406 includes a selective compliance assembly robot arm (SCARA) 1408 coupled to a column 1410 by a prismatic joint 1412. The prismatic joints 1412 are configured to translate along the column 1410 (e.g., along rails 1414) to allow each of the handles 1402 to be translated in the z-direction, providing a first degree of freedom. The SCARA arm 1408 is configured to allow motion of the handle 1402 in an x-y plane, providing two additional degrees of freedom.

In some embodiments, one or more load cells are positioned in the controller 1400. For example, in some embodiments, a load cell (not shown) is positioned in the body of each of the gimbals 1404. By providing a load cell, portions of the controller 1400 are capable of operating under admittance control, thereby advantageously reducing the perceived inertia of the controller 1400 while in use. In some embodiments, the positioning platform 1406 is configured for admittance control, while the gimbal 1404 is configured for impedance control. In other embodiments, the gimbal 1404 is configured for admittance control, while the positioning platform 1406 is configured for impedance control. Accordingly, for some embodiments, the translational or positional degrees of freedom of the positioning platform 1406 can rely on admittance control, while the rotational degrees of freedom of the gimbal 1404 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

FIG. 15 is a block diagram illustrating a localization system 1500 that estimates a location of one or more elements of the robotic system, such as the location of the instrument, in accordance to an example embodiment. The localization system 1500 may be a set of one or more computer devices configured to execute one or more instructions. The computer devices may be embodied by a processor (or processors) and computer-readable memory in one or more components discussed above. By way of example and not limitation, the computer devices may be in the tower 112 shown in FIG. 1, the cart 102 shown in FIGS. 1-3B, the beds shown in FIGS. 4-9, etc.

As shown in FIG. 15, the localization system 1500 may include a localization module 1502 that processes input data 1504a, 1504b, 1504c, and 1504d to generate location data 1506 for the distal tip of a medical instrument. The location data 1506 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 1504a-d are now described in greater detail. Pre-operative mapping may be accomplished through the use of the collection of low dose CT scans. Pre-operative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 1504a (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 1504b. The localization module 1502 may process the vision data 1504b to enable one or more vision-based location tracking. For example, the preoperative model data may be used in conjunction with the vision data 1504b to enable computer vision-based tracking of the medical instrument (e.g., an endoscope or an instrument advance through a working channel of the endoscope). For example, using the preoperative model data 1504a, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 1502 may identify circular geometries in the preoperative model data 1504a that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 1504b to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 1502 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 1504c. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 1504d may also be used by the localization module 1502 to provide localization data 1506 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 15 shows, a number of other input data can be used by the localization module 1502. For example, although not shown in FIG. 15, an instrument utilizing shape-sensing fiber can provide shape data that the localization module 1502 can use to determine the location and shape of the instrument.

The localization module 1502 may use the input data 1504a-d in combination(s). In some cases, such a combination may use a probabilistic approach where the localization module 1502 assigns a confidence weight to the location determined from each of the input data 1504a-d. Thus, where the EM data 1504c may not be reliable (as may be the case where there is EM interference) the confidence of the location determined by the EM data 1504c can be decrease and the localization module 1502 may rely more heavily on the vision data 1504b and/or the robotic command and kinematics data 1504d.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Introduction

Embodiments of this disclosure relate to robotic systems having modular tool drivers. The robotic surgical system may include a stationary instrument driver comprising a first instrument driver body attached to a distal joint of a first robotic arm and operable to drive one or more functions of a first surgical tool. The robotic surgical system may also include a mobile instrument driver comprising an instrument driver carriage translatable along a longitudinal base and operable to drive one or more functions of a mobile surgical tool.

### 3. Description

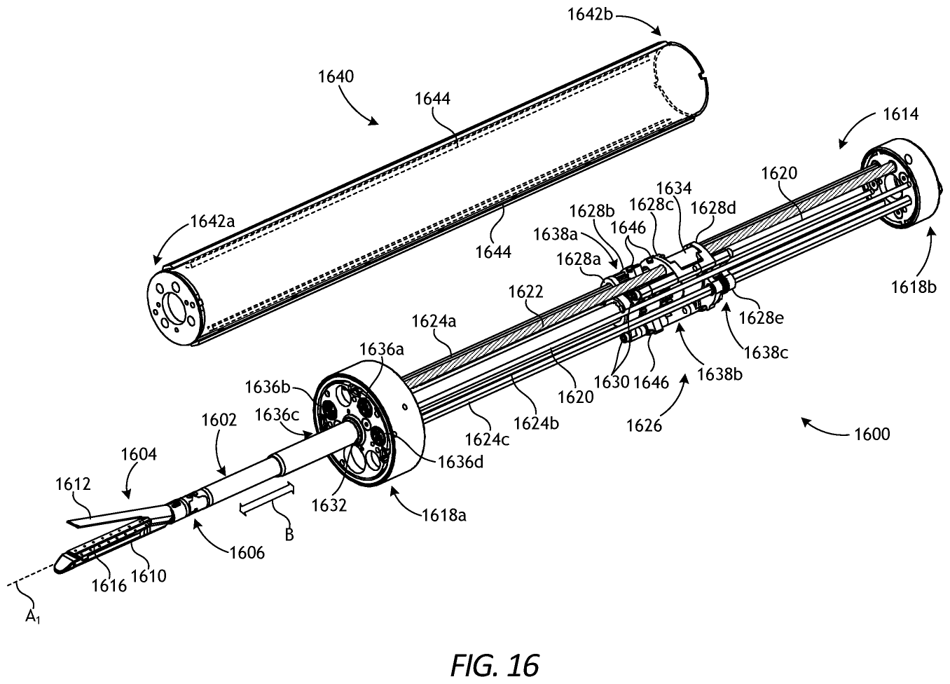

FIG. 16 is an isometric side view of an example surgical tool 1600 that may incorporate some or all of the principles of the present disclosure. The surgical tool 1600 may be similar in some respects to any of the medical instruments described above with reference to FIGS. 11-13 and, therefore, may be used in conjunction with a robotic surgical system, such as the robotically-enabled systems 100, 400, and 900 of FIGS. 1-13. As illustrated, the surgical tool 1600 includes an elongated shaft 1602, an end effector 1604 arranged at the distal end of the shaft 1602, and an articulable wrist 1606 (alternately referred to as a “wrist joint”) that interposes and couples the end effector 1604 to the distal end of the shaft 1602.

The terms “proximal” and “distal” are defined herein relative to a robotic surgical system having an interface configured to mechanically and electrically couple the surgical tool 1600 to a robotic manipulator. The term “proximal” refers to the position of an element closer to the robotic manipulator and the term “distal” refers to the position of an element closer to the end effector 1604 and thus closer to the patient during operation. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

The surgical tool 1600 can have any of a variety of configurations capable of performing one or more surgical functions. In the illustrated embodiment, the end effector 1604 comprises a surgical stapler, alternately referred to as an “endocutter,” configured to cut and staple (fasten) tissue. As illustrated, the end effector 1604 includes opposing jaws 1610, 1612 configured to move (articulate) between open and closed positions. Alternatively, the end effector 1604 may comprise other types of instruments having opposing jaws such as, but not limited to, tissue graspers, surgical scissors, advanced energy vessel sealers, clip appliers, needle drivers, a babcock including a pair of opposed grasping jaws, bipolar jaws (e.g., bipolar Maryland grasper, forceps, a fenestrated grasper, etc.), etc. In other embodiments, the end effector 1604 may instead comprise any end effector or instrument capable of being operated in conjunction with the presently disclosed robotic surgical systems and methods. Such end effectors or instruments include, but are not limited to, a suction irrigator, an endoscope (e.g., a camera), or any combination thereof.

One or both of the jaws 1610, 1612 may be configured to pivot to actuate the end effector 1604 between open and closed positions. In the illustrated example, the second jaw 1612 is rotatable (pivotable) relative to the first jaw 1610 to move between an open, unclamped position and a closed, clamped position. In other embodiments, however, the first jaw 1610 may move (rotate) relative to the second jaw 1612, without departing from the scope of the disclosure. In yet other embodiments, both jaws 1610, 1612 may move to actuate the end effector 1604 between open and closed positions.

In the illustrated example, the first jaw 1610 is referred to as a “cartridge” or “channel” jaw, and the second jaw 1612 is referred to as an “anvil” jaw. The first jaw 1610 may include a frame that houses or supports a staple cartridge, and the second jaw 1612 is pivotally supported relative to the first jaw 1610 and defines a surface that operates as an anvil to deform staples ejected from the staple cartridge during operation.

The wrist 1606 enables the end effector 1604 to articulate (pivot) relative to the shaft 1602 and thereby position the end effector 1604 at various desired orientations and locations relative to a surgical site. In the illustrated embodiment, the wrist 1606 is designed to allow the end effector 1604 to pivot (swivel) left and right relative to a longitudinal axis A₁ of the shaft 1602. In other embodiments, however, the wrist 1606 may be designed to provide multiple degrees of freedom, including one or more translational variables (i.e., surge, heave, and sway) and/or one or more rotational variables (i.e., Euler angles or roll, pitch, and yaw). The translational and rotational variables describe the position and orientation of a component of a surgical system (e.g., the end effector 1604) with respect to a given reference Cartesian frame. “Surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

In the illustrated embodiment, the pivoting motion at the wrist 1606 is limited to movement in a single plane, e.g., only yaw movement relative to the longitudinal axis A₁. The end effector 1604 is depicted in FIG. 16 in the unarticulated position where the longitudinal axis of the end effector 1604 is substantially aligned with the longitudinal axis A₁ of the shaft 1602, such that the end effector 1604 is at a substantially zero angle relative to the shaft 1602. In the articulated position, the longitudinal axis of the end effector 1604 would be angularly offset from the longitudinal axis A₁ such that the end effector 1604 would be oriented at a non-zero angle relative to the shaft 1602.

Still referring to FIG. 16, the surgical tool 1600 may include a drive housing or “handle” 1614 that operates as an actuation system designed to facilitate articulation of the wrist 1606 and actuation (operation) of the end effector 1604 (e.g., clamping, firing, rotation, articulation, energy delivery, etc.). As described in more detail below, the drive housing 1614 provides various coupling features that releasably couple the surgical tool 1600 to an instrument driver of a robotic surgical system.

The drive housing 1614 includes a plurality of drive members (obscured in FIG. 16) that extend to the wrist 1606 and the end effector 1604. Selective actuation of one or more of the drive members causes the end effector 1604 to articulate (pivot) relative to the shaft 1602 at the wrist 1606. Selective actuation of one or more other drive members causes the end effector 1604 to actuate (operate). Actuating the end effector 1604 may include closing and/or opening the jaws, 1610, 1612, and thereby enabling the end effector 1604 to grasp (clamp) onto tissue. Once tissue is grasped or clamped between the opposing jaws 1610, 1612, actuating the end effector 1604 may further include “firing” the end effector 1604, which may refer to causing a cutting element or knife (not visible) to advance distally within a slot 1616 defined in the first jaw 1610. As it moves distally, the cutting element transects any tissue grasped between the opposing jaws 1610, 1612. Moreover, as the cutting element advances distally, a plurality of staples contained within the staple cartridge (e.g., housed within the first jaw 1610) are urged (cammed) into deforming contact with corresponding anvil surfaces (e.g., pockets) provided on the second jaw 1612. The deployed staples may form multiple rows of staples that seal opposing sides of the transected tissue.

As illustrated, the drive housing 1614 has a first or “distal” end 1618a and a second or “proximal” end 1618b opposite the first end 1618a (alternately referred to as the “drive housing”). In some embodiments, one or more struts 1620 (two shown) extend longitudinally between the first and second ends 1618a,b to help fix the distance between the first and second ends 1618a,b, provide structural stability to the drive housing 1614, and secure the first end 1618a to the second end 1618b. In other embodiments, however, the struts 1620 may be omitted, without departing from the scope of the disclosure.

The drive housing 1614 may also include a lead screw 1622 and one or more splines 1624, which also extend longitudinally between the first and second ends 1618a,b. In the illustrated embodiment, the drive housing 1614 includes a first spline 1624a, a second spline 1624b, and a third spline 1624c. While three splines 1624a-c are depicted in the drive housing 1614, more or less than three may be included, without departing from the scope of the disclosure. Unlike the struts 1620, the lead screw 1622 and the splines 1624a-c are rotatably mounted to the first and second ends 1618a,b. As described in more detail below, selective rotation of the lead screw 1622 and the splines 1624a-c causes various functions of the drive housing 1614 to transpire, such as translating the end effector 1604 along the longitudinal axis A₁ (e.g., z-axis translation) causing the end effector 1604 to articulate (pivot) at the wrist 1606, causing the jaws 1610, 1612 to open and close, and causing the end effector 1604 to fire (operate).

The drive housing 1614 further includes a carriage 1626 movably mounted along the lead screw 1622 and the splines 1624a-c and housing various activating mechanisms configured to cause operation of specific functions of the end effector 1604. The carriage 1626 may comprise two or more layers, shown in FIG. 16 as a first layer 1628a, a second layer 1628b, a third layer 1628c, a fourth layer 1628d, and a fifth layer 1628e. The lead screw 1622 and the splines 1624a-c each extend through portions of one or more of the layers 1628a-e to allow the carriage 1626 to translate along the longitudinal axis A₁ with respect to the lead screw 1622 and the splines 1624a-c. In some embodiments, the layers 1628a-e may be secured to each other in series using one or more mechanical fasteners 1630 (two visible) extending between the first layer 1628a and the fifth layer 1628e and through coaxially aligned holes defined in some or all of the layers 1628a-e. While five layers 1628a-e are depicted, more or less than five may be included in the carriage 1626, without departing from the scope of the disclosure.

The shaft 1602 is coupled to and extends distally from the carriage 1626 through the first end 1618a (alternately referred to as the “drive housing”) of the drive housing 1614. In the illustrated embodiment, for example, the shaft 1602 penetrates the first end 1618a at a central aperture 1632 defined through the first end 1618a. The carriage 1626 is movable between the first and second ends 1618a,b along the longitudinal axis A₁ (e.g., z-axis translation) and is thereby able to advance or retract the end effector 1604 relative to the drive housing 1614, as indicated by the arrows B. More specifically, in some embodiments, the carriage 1626 includes a carriage nut 1634 mounted to the lead screw 1622 and secured between the third and fourth layers 1628c,d. The outer surface of the lead screw 1622 defines outer helical threading and the carriage nut 1634 defines corresponding internal helical threading (not shown) matable with the outer helical threading of the lead screw 1622. As a result, rotation of the lead screw 1622 causes the carriage nut 1634 to advance or retract the carriage 1626 along the longitudinal axis A₁ and correspondingly advance or retract the end effector 1604 relative to the drive housing 1614.

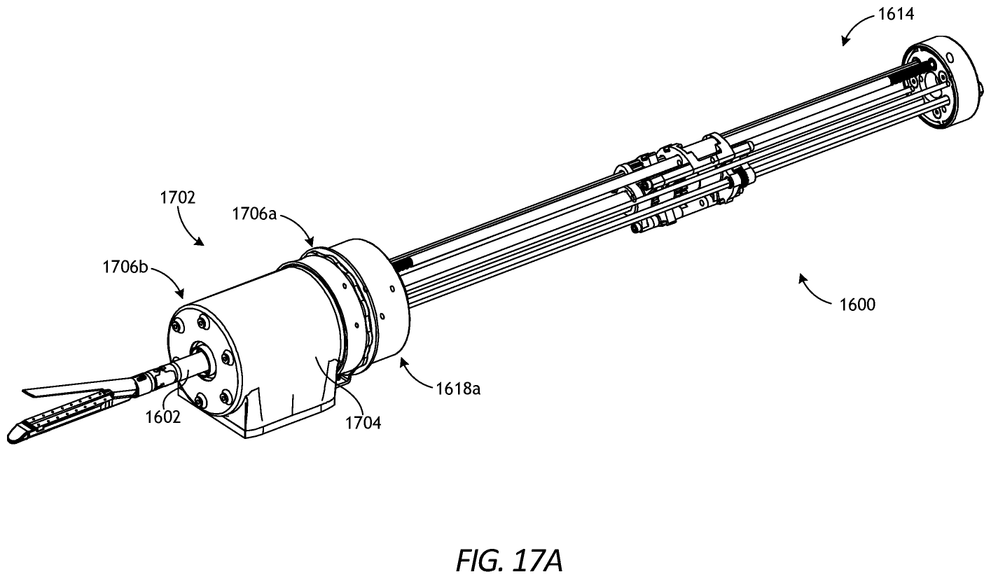

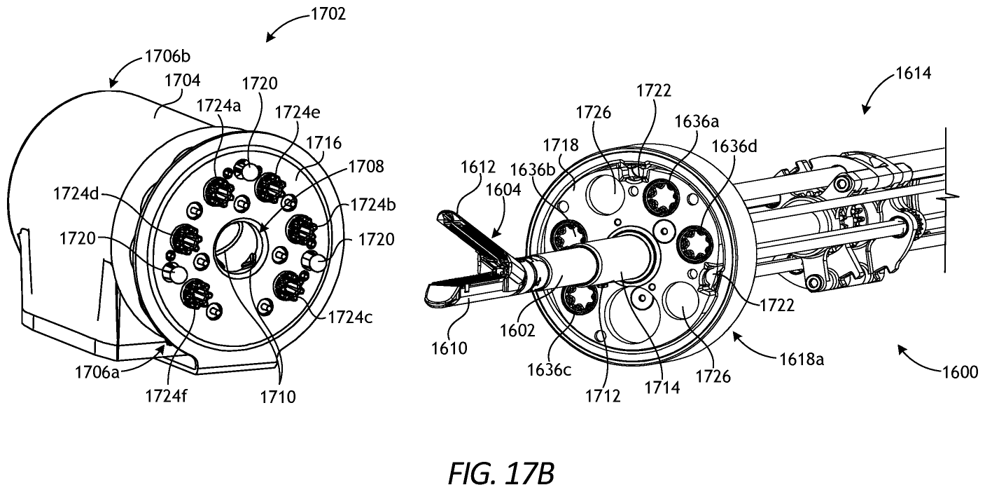

As indicated above, the lead screw 1622 and the splines 1624a-c are rotatably mounted to the first and second ends 1618a,b. More specifically, the first end 1618a of the drive housing 1614 may include one or more rotatable drive inputs actuatable to independently drive (rotate) the lead screw 1622 and the splines 1624a-c. In the illustrated embodiment, the drive housing 1614 includes a first drive input 1636a, a second drive input 1636b, a third drive input 1636c (occluded by the shaft 1602, see FIG. 17B), and a fourth drive input 1636d. As described below, each drive input 1636a-d may be matable with a corresponding drive output of an instrument driver such that movement (rotation) of a given drive output correspondingly moves (rotates) the associated drive input 1636a-d and thereby rotates the mated lead screw 1622 or spline 1624a-c. While only four drive inputs 1636a-d are depicted, more or less than four may be included in the drive housing 1614, depending on the application.

The first drive input 1636a may be operatively coupled to the lead screw 1622 such that rotation of the first drive input 1636a correspondingly rotates the lead screw 1622, which causes the carriage nut 1634 and the carriage 1626 to advance or retract along the longitudinal axis A₁, depending on the rotational direction of the lead screw 1622. As used herein the phrase “operatively coupled” refers to a coupled engagement, either directly or indirectly, where movement of one component causes corresponding movement of another component. With respect to the first drive input 1636a being operatively coupled to the lead screw 1622, such operative coupling may be facilitated through intermeshed gears (not shown) arranged within the second end 1618a, but could alternatively be facilitated through other mechanical means, such as cables, pulleys, drive rods, direct couplings, etc., without departing from the scope of the disclosure.

The second drive input 1636b may be operatively coupled to the first spline 1624a such that rotation of the second drive input 1636b correspondingly rotates the first spline 1624a. In some embodiments, the first spline 1624a may be operatively coupled to a first activating mechanism 1638a of the carriage 1626, and the first activating mechanism 1638a may be operable to open and close the jaws 1610, 1612. Accordingly, rotating the second drive input 1636b will correspondingly actuate the first activating mechanism 1638a and thereby open or close the jaws 1610, 1612, depending on the rotational direction of the first spline 1624a.

The third drive input 1636c may be operatively coupled to the second spline 1624b such that rotation of the third drive input 1636c correspondingly rotates the second spline 1624b. In some embodiments, the second spline 1624b may be operatively coupled to a second activating mechanism 1638b of the carriage 1626, and the second activating mechanism 1638b may be operable to articulate the end effector 1604 at the wrist 1606. Accordingly, rotating the third drive input 1636c will correspondingly actuate the second activating mechanism 1638b and thereby cause the wrist 1606 to articulate in at least one degree of freedom, depending on the rotational direction of the second spline 1624b.

The fourth drive input 1636d may be operatively coupled to the third spline 1624c such that rotation of the fourth drive input 1636d correspondingly rotates the third spline 1624c. In some embodiments, the third spline 1624c may be operatively coupled to a third activating mechanism 1638c of the carriage 1626, and the third activating mechanism 1638c may be operable to fire the cutting element (knife) at the end effector 1604. Accordingly, rotating the fourth drive input 1636d will correspondingly actuate the third activating mechanism 1638c and thereby cause the knife to advance or retract, depending on the rotational direction of the third spline 1624c.

In the illustrated embodiment, and as described in more detail below, the activating mechanisms 1838a-c comprise intermeshed gearing assemblies including one or more drive gears driven by rotation of the corresponding spline 1624a-c and configured to drive one or more corresponding driven gears that cause operation of specific functions of the end effector 1604.

In some embodiments, the drive housing 1614 may include a shroud 1640 sized to receive and otherwise surround the carriage 1626, the lead screw 1622, and the splines 1624a-c. In the illustrated embodiment, the shroud 1640 comprises a tubular or cylindrical structure having a first end 1642a matable with the first end 1618a of the drive housing 1614, and a second end 1642b matable with the second end 1618b of the drive housing 1614. The carriage 1626, the lead screw 1622, and the splines 1624a-c can all be accommodated within the interior of the shroud 1640, and the carriage 1626 may engage and traverse (ride on) one or more rails 1644 (shown in phantom) fixed to the shroud 1640. The rails 1644 extend longitudinally and parallel to the lead screw 1622 and are sized to be received within corresponding notches 1646 defined on the outer periphery of the carriage 1626 and, more particularly, on the outer periphery of one or more of the carriage layers 1628a-e. As the carriage 1626 translates along the longitudinal axis A₁, the rails 1644 help maintain the angular position of the carriage 1626 and assume any torsional loading that might otherwise adversely affect movement or operation of the carriage 1626.

FIG. 17A is an isometric view of the surgical tool 1600 of FIG. 16 releasably coupled to an example instrument driver 1702 according to one or more embodiments. The instrument driver 1702 may be similar in some respects to the instrument drivers 1102, 1200 of FIGS. 11 and 12, respectively, and therefore may be best understood with reference thereto. Similar to the instrument drivers 1102, 1200, for example, the instrument driver 1702 may be mounted to or otherwise positioned at the end of a robotic arm (not shown) and is designed to provide the motive forces required to operate the surgical tool 1600. Unlike the instrument drivers 1102, 1200, however, the shaft 1602 of the surgical tool 1600 extends through and penetrates the instrument driver 1702.

The instrument driver 1702 has a body 1704 having a first or “proximal” end 1706a and a second or “distal” end 1706b opposite the first end 1706a. In the illustrated embodiment, the first end 1706a of the instrument driver 1702 is matable with and releasably coupled to the first end 1618a of the drive housing 1614, and the shaft 1602 of the surgical tool 1600 extends through the body 1704 and distally from the second end 1706b.

FIG. 17B depicts separated isometric end views of the instrument driver 1702 and the surgical tool 1600 of FIG. 17A. With the jaws 1610, 1612 closed, the shaft 1602 and the end effector 1604 can penetrate the instrument driver 1702 by extending through a central aperture 1708 defined longitudinally through the body 1704 between the first and second ends 1706a,b. To align the surgical tool 1600 with the instrument driver 1702 in a proper angular orientation, one or more alignment guides 1710 may be provided or otherwise defined within the central aperture 1708 and configured to engage one or more corresponding alignment features 1712 provided on the surgical tool 1600. In the illustrated embodiment, the alignment feature 1712 comprises a protrusion or projection defined on or otherwise provided by an alignment nozzle 1714 extending distally from the first end 1618a of the drive housing 1614. In one or more embodiments, the alignment guide 1710 may comprise a curved or arcuate shoulder or lip configured to receive and guide the alignment feature 1712 as the alignment nozzle 1714 enters the central aperture 1708. As a result, the surgical tool 1600 is oriented to a proper angular alignment with the instrument driver 1702 as the alignment nozzle 1714 is advanced distally through the central aperture 1708. In other embodiments, the alignment nozzle 1714 may be omitted and the alignment feature 1712 may alternatively be provided on the shaft 1602, without departing from the scope of the disclosure.

As illustrated, a drive interface 1716 is provided at the first end 1706a of the instrument driver 1702, and a driven interface 1718 is provided at the first end 1618a of the drive housing 1614. The drive and driven interfaces 1716, 1718 may be configured to mechanically, magnetically, and/or electrically couple the drive housing 1614 to the instrument driver 1702. To accomplish this, the drive and driven interfaces 1716, 1718 may provide one or more matable locating features configured to secure the drive housing 1614 to the instrument driver 1702. In the illustrated embodiment, for example, the drive interface 1716 provides one or more interlocking features 1720 (three shown) configured to locate and mate with one or more complementary-shaped pockets 1722 (two shown, one occluded) provided on the driven interface 1718. In some embodiments, the features 1720 may be configured to align and mate with the pockets 1722 via an interference or snap fit engagement, for example.

The instrument driver 1702 also includes one or more drive outputs that extend through the drive interface 1716 to mate with the drive inputs 1636a-d provided at the first end 1618a of the drive housing 1614. More specifically, the instrument driver 1702 includes a first drive output 1724a matable with the first drive input 1636a, a second drive output 1724b matable with the second drive input 1636b, a third drive output 1724b matable with the third drive input 1636c, and a fourth drive output 1724d matable with the fourth drive input 1636d. In some embodiments, as illustrated, the drive outputs 1724a-d may define splines or features designed to mate with corresponding splined receptacles of the drive inputs 1636a-d. Once properly mated, the drive inputs 1636a-d will share axes of rotation with the corresponding drive outputs 1724a-d to allow the transfer of rotational torque from the drive outputs 1724a-d to the corresponding drive inputs 1636a-d. In some embodiments, each drive output 1724a-d may be spring loaded and otherwise biased to spring outwards away from the drive interface 1716. Each drive output 1724a-d may be capable of partially or fully retracting into the drive interface 1716.

In some embodiments, the instrument driver 1702 may include additional drive outputs, depicted in FIG. 17B as a fifth drive output 1724e and a sixth drive output 1724f. The fifth and sixth drive outputs 1724e,f may be configured to mate with additional drive inputs (not shown) of the drive housing 1614 to help undertake one or more additional functions of the surgical tool 1600. In the illustrated embodiment, however, the drive housing 1614 does not include additional drive inputs matable with the fifth and sixth drive outputs 1724e,f. Instead, the driven interface 1718 defines corresponding recesses 1726 configured to receive the fifth and sixth drive outputs 1724e,f. In other applications, however, fifth and/or sixth drive inputs (not shown) could be included in the drive housing 1614 to mate with the fifth and sixth drive outputs 1724e,f, or the surgical tool 1600 might be replaced with another surgical tool having fifth and/or sixth drive inputs, which would be driven by the fifth and/or sixth drive outputs 1724e,f.

While not shown, in some embodiments, an instrument sterile adapter (ISA) may be placed at the interface between the instrument driver 1702 and the surgical tool 1600. In such applications, the interlocking features 1720 may operate as alignment features and possible latches for the ISA to be placed, stabilized, and secured. Stability of the ISA may be accomplished by a nose cone feature provided by the ISA and extending into the central aperture 1708 of the instrument driver 1702. Latching can occur either with the interlocking features 1720 or at other locations at the interface. In some cases, the ISA will provide the means to help align and facilitate the latching of the surgical tool 1600 to the ISA and simultaneously to the instrument driver 1702.

### Multi Tool Drive Robotic Configurations

In accordance with the present disclosure, robotic surgical systems may be configured for removably attaching instrument drivers of varying architecture. For example, such modular robotic systems are configured to use instrument drivers and compatible surgical tools of a first type, wherein the instrument driver is stationary and shaft insertion is through the instrument driver, as well as instrument drivers of a second type, wherein the instrument driver is mobile on a stage/base. This modular robotic surgical system configuration provides for a wider selection of instrument drivers and compatible surgical tools for use in robotic surgery. The drivers and tools may be selected by the user (e.g., physician, nurse, operator, etc.) and removably attached to the robotic surgical system based on instrument driver and tool availability, familiarity preferences, and surgical strategy, among other considerations. The modularity also allows for robotic arms to be optimized structurally, thus potentially utilizing more robust robotic arms for heavier instrument drivers in order to provide increased stability.

In preparation for a surgery, and prior to draping, a robotic surgical system is mechanically set-up for a particular procedure or set of surgical procedures, e.g., laparoscopy. In the mechanical set-up, modular components, e.g., instrument drivers and robotic arms with modular bases, described in greater detail below, are removably connected to the robotic surgical system as desired. This is particularly useful in facilities (e.g., hospitals, clinics, etc.) with a limited number of robotic surgical systems. For example, if a facility has only one robot, and three different procedures are planned for the day, a physician may, during setup, mount the varying instrument drivers needed for the day to the robotic surgical system and drape. In some embodiments, a surgical tool coupled to an instrument driver may be withdrawn from the operation site during surgery and the instrument driver (and surgical tool) may be replaced. That is, an instrument driver of one driver type may be switched with an instrument driver of another driver type for use with a particular surgical tool compatible with a certain type of instrument driver.

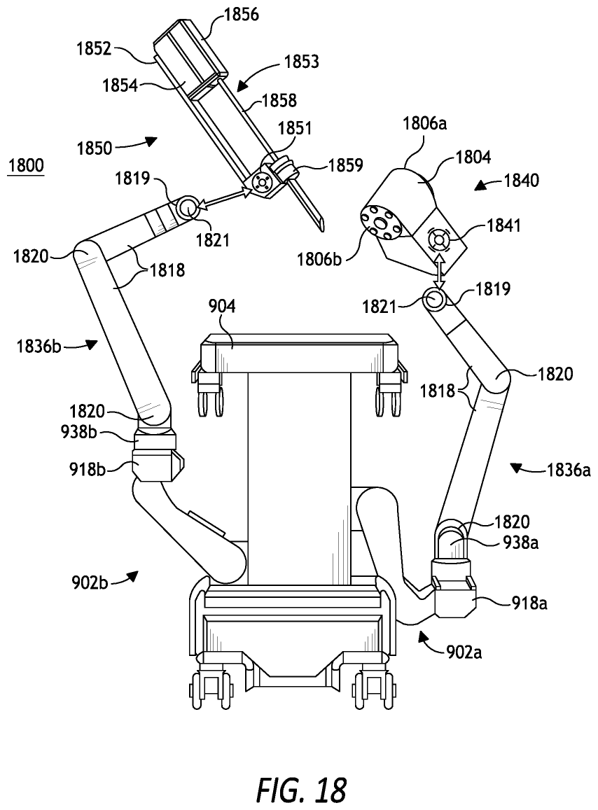

FIG. 18 illustrates an end view of an example robotic surgical system 1800, according to one or more embodiments of the disclosure. The robotic system 1800 may be similar in some respects to the robotic system 900 of FIGS. 9A-9C and therefore may be best understood with reference thereto, where similar reference numerals will correspond to similar components not described again in detail.

As illustrated, the robotic system 1800 includes the two adjustable arm supports 902a and 902b mounted on opposite sides of the table 904. A first robotic arm 1836a is attached to the first bar or rail 918a of the first adjustable arm support 902a. The first robotic arm 936a includes a base 938a attached to the first rail 918a. Similarly, a second robotic arm 1836b includes a base 938b attached to the second rail 918b. Each robotic arm comprises a series of linkages 1818 connected by a corresponding series of j oints 1820. Each joint 1820 includes an independent actuator, and each actuator includes an independently controllable motor. Each independently controllable joint 1820 represents an independent degree of freedom available to the corresponding robotic arm 936a, 936b. While two linkages 1818 are illustrated for each arm 936a, 936b, it is to be appreciated that either of the robotic arms 936a,b may include more or less linkages 1818 and corresponding joints 1820, thus having any number of degrees of freedom.

At least one joint 1820 of the robotic arms, 1836a, 1836b, is a modular joint 1819 configured to removably attach, and electronically couple to, an instrument driver e.g., an instrument driver of a first type 1840 and an instrument driver of a second type 1850. In some embodiments, the modular joint 1819 may removably attach to additional linkages 1818. The modular joints 1819 are illustrated as positioned at the distal end the first robotic arm 1836a and second robotic arm 1836b. However, it is to be appreciated that each robotic arm 1836a, 1836b may have more than one modular joint 1819. That is, any of the joints 1820 movably connecting linkages 1818 together in series may comprise a modular joint 1819 configured to removably connect to an instrument driver 1840, 1850, allowing one to connect a modular instrument driver at any location along the robotic arm 1836a,b.

The modular joint 1819 may include a modular interface 1821 configured to both mechanically attach and electronically couple the robotic arm 1836a,b to an instrument driver 1840, 1850 designed to manipulate a medical instrument (“surgical tool”). The modular interface 1821 allows for the instrument driver 1840, 1850 to be easily attached and/or removed during the mechanical setup process by a medical professional or technician. In some embodiments, the modular interface 1821 directly connects to an instrument driver 1840, 1850 via an instrument driver interface 1841, 1851, respectively. To accomplish this, the modular interface 1821 and instrument driver interface 1841, 1851 may provide one or more matable features configured to attach the instrument driver 1820 to the modular joint 1819 and facilitate electrical and power communication therebetween. In other embodiments, an adaptor (not illustrated) may be used to facilitate mechanical and electronic attachment to an instrument driver 1840, 1850. For example, if the instrument driver 1850 has an instrument driver interface 1851 that is not directly compatible, mechanically and/or electronically, with the modular interface 1821, an adaptor having an interface compatible with the instrument driver interface 1851 on one end and an interface compatible with the modular interface 1821 on the other, may be used to attach the otherwise incompatible instrument driver 1850 to the robotic arm 1836a,b. A sensor (not illustrated) in the modular interface 1821, may be configured to read and determine the type of instrument driver 1840, 1850, attached. The electronically coupled computer-based control system is thus configured to control any attached instrument driver 1840, 1850. That is, a single computer system may drive multiple types of instrument drivers 1840, 1850.

Mating the modular joint 1819 and instrument driver 1840, 1850 may be a pivotal attachment. That is, the robotic arm 936a,b may be controlled to rotate the connected instrument driver 1840, 1850 about the modular joint 1819. This allows for the connection between the modular joint 1819 and instrument driver 1840, 1850 to introduce a degree of freedom to the arm 936a,b of the robotic system 1800. In other embodiments, the mating of the modular joint 1819 and instrument driver 1840, 1850 is fixed, such that the connection does not allow for the instrument driver 1840, 1850 to move with respect to the modular joint 1819.

As briefly stated above, the modular configuration of the robotic surgical system 1800 allows for the connection of instrument drivers 1840, 1850 of various types. For example, illustrated in FIG. 18 is an instrument driver of a first type, 1840, that may be an instrument driver similar to the instrument driver 1702 of FIGS. 17A-B, and therefore may be best understood with reference thereto. The instrument driver of the first type 1840 may be removably mounted to the modular joint 1819 and is designed to provide the motive forces required to operate a coupled surgical tool, such as surgical tool 1600. The instrument driver of the first type 1840, has a body 1804 with first end 1806a that is matable with and releasably coupled to the first end 1618a (FIGS. 17A-17B) of the drive housing 1614 (FIGS. 17A-17B), and the shaft 1602 (FIGS. 17A-17B) of the surgical tool 1600 (FIGS. 17A-17B) extends through the body 1804 and distally from a second end 1806b. The body 1804 of the instrument driver of the first type 1840 may pivot or rotate with respect to the modular joint 1819 but is otherwise at a substantially fixed distance from the modular joint 1819. That is, the large mass of the body 1804 that houses the various drive components, motors, actuators, etc., is movably secured at a set distance from the modular joint 1819. Briefly summarizing, instrument drivers of a first type 1840 may be generally characterized as a stationary instrument drivers that drive shaft insertion through the body 1804 of the instrument driver 1840. In a stationary instrument driver, insertion of an instrument shaft can, for example, be controlled by one or more drive outputs of a drive interface 1716 (FIG. 17B), or by motion of an arm supporting the instrument driver (e.g., along a virtual rail as described previously).

Also illustrated in FIG. 18 is an instrument driver of a second type 1850, that may include a base (or “stage”) 1852, including a longitudinal track and an instrument driver carriage 1854 which is slidingly engaged with the longitudinal base 1852. As used herein, “sliding” engagement or “slidingly engaged” can include rolling contact, slipping contact, or other forms of engagement that permit relative translation between the slidingly engaged members. The base 1852 may be configured to couple mechanically and electronically to a modular joint 1819 of a robotic arm 1836b at the interface 1851 such that articulation of the robotic arm 1836b positions and/or orients the instrument driver of the second type 1850 in space. Additionally, the instrument driver carriage 1854 may be configured to couple to a surgical instrument 1853 via an instrument base 1856. An instrument shaft 1858 extends distally from the instrument base 1856 and includes an end effector (not illustrated) disposed at a distal end thereof. Generally, the instrument driver carriage 1854 may be actuated along the longitudinal base 1852 to axially position the instrument shaft 1858 within the optionally attached cannula 1859 and thus, enable positioning of the end effector within a surgical workspace within the patient. Additionally, the instrument base 1856 may be decoupled from the instrument driver carriage 1854 to exchange with another tool, such as another tool having an end effector with different functionality. In a mobile instrument driver, insertion of an instrument shaft can, for example, be controlled through movement of a stage/base along a track.

Generally, the instrument driver carriage 1854 may additionally be configured to orient and/or actuate the end effector of the attached surgical instrument 1853. For example, the instrument driver carriage 1854 may enable rotation of the tool shaft 1858 around a longitudinal tool axis, thereby rotating the end effector of the surgical instrument 1853 about the longitudinal tool axis. Additionally, the instrument driver carriage 1854 may actuate specific functionalities of the end effector, such as through one or more internal instrument outputs in communication with activating mechanisms in the surgical instrument 1853 including and without limitation a cable system manipulated and controlled by actuated drives (e.g., linear axis drive, rotary axis drive, etc. such as those described herein). The instrument driver carriage 1854 may include different configurations of actuated drives. Such instruments and instrument drivers are described in greater detail in U.S. Patent Publication No. 2018/0116738 entitled Tool Driver with Rotary Drives for Use in Robotic Surgery. In other words, instrument drivers of a second type 1850 may be generally characterized as a mobile instrument drivers that drive an associated tool from a proximal end to a distal end of the base 1852. Instead of having a relatively stationary body 1804 that houses the various drive motors pivotally attached to the joint 1819, the instrument drivers of the second type 1850 have a drive carriage 1854 that houses the motors for instrument driving that is longitudinally moveable along a base 1852 that is pivotally attached to the modular joint 1819.

### Multi Arm Robotic Configurations

In accordance with the present disclosure, robotic surgical systems may be configured for removably attaching robotic arms of varying structural configurations and architecture, including instrument drivers of varying architecture. For example, such a modular robotic system is configured to use robotic arms configured for use with instrument drivers of a first type e.g., instrument drivers 1702 (FIGS. 17A-17B) and 1840, wherein shaft insertion is through the instrument driver, and of a second type e.g., 1850, wherein the instrument driver is mobile on a stage. This modular robotic surgical system configuration provides for a wider selection of instrument drivers of various functions, weights, operating torque and the like with an associated robotic arm that is structurally configured to meet the load requirements of the instrument driver type and associated surgical tool. The robotic arms, instrument drivers, and surgical tools may be selected by the physician and removably attached to the robotic system based on instrument driver and tool availability, familiarity preferences, and surgical strategy, among other considerations.

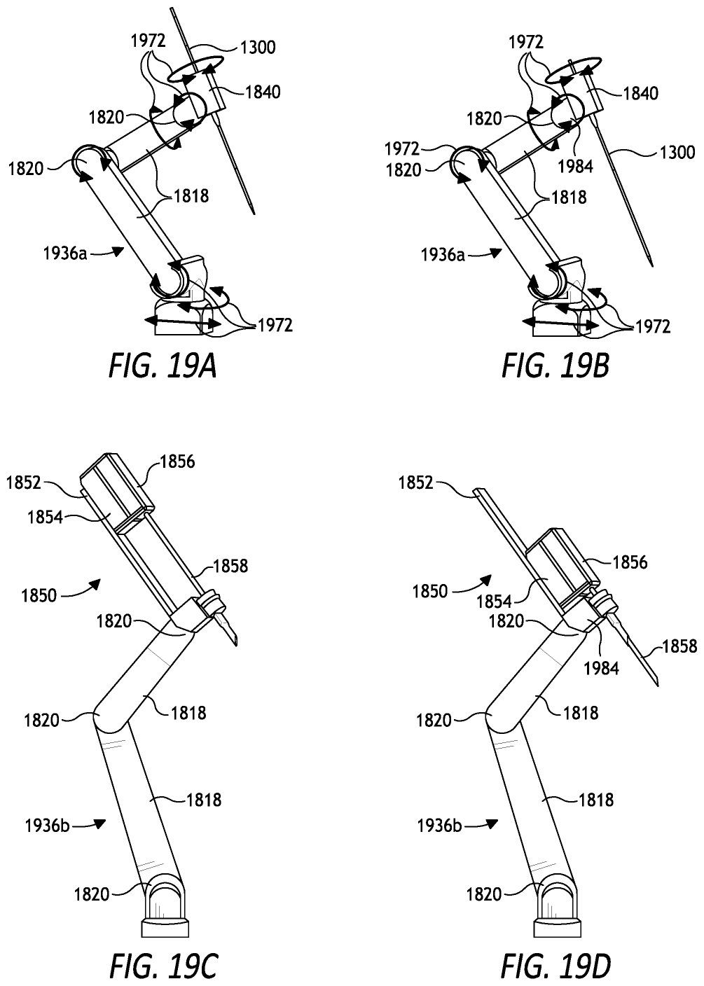

FIGS. 19A-D illustrate instrument drivers of different types with coupled surgical instruments in fully proximal and fully distal positions attached to a robotic arm. In particular, FIGS. 19A and 19B, illustrate an instrument driver of a first type 1840 located on a robotic arm 1936a having a plurality of linkages 1818 and various degrees of freedom 1972 associated with each joint 1820. The instrument driver of a first type 1840, other than having a rotational degree of freedom with the corresponding joint, is stationary. The change in the position of the center of mass of the instrument driver 1840 and coupled surgical tool combination, e.g. surgical tool 1300 of FIG. 13, is minimal between the fully proximal position illustrated in FIG. 19A to the fully distal position illustrated in 19B, since the mass of the shaft in relation to the mass of the stationary instrument driver 1840 is fairly negligible. This mass centralization property of robotic arms 1936a with instrument drivers of a first type 1840 allows for the arms to be optimized as lightweight and nimble.

FIGS. 19C and 19D, illustrate an instrument driver of the second type 1850 located on a robotic arm 1936b having a plurality of linkages 1818 and various degrees or freedom associated with each joint 1820. The instrument driver carriage 1854 houses the drive components (e.g., motors) for driving various functions of the coupled surgical tool 1856. The instrument driver carriage 1854 is mobile on the base 1852. Thus, there is a significant change in the position of the center of mass of the instrument driver carriage 1854 and attached surgical tool 1856 combination between the fully proximal position illustrated in FIG. 19C and the fully distal position illustrated in FIG. 19D. That is, in the fully proximal position illustrated in FIG. 19C, the large mass of the instrument driver carriage 1854 is located at a distance away from the connection joint 1820. The force of gravity acting on the fully proximal instrument driver carriage 1854 generates a moment of force acting on the joint 1820 that is proportional to a distance from the point of rotation. The further distal the instrument driver carriage 1854, the greater the moment of force acting on the instrument driver of the second type 1850, potentially providing instabilities to the arm 1936b and instrument driver 1850. Said another way, the base/stage 1852 is a lever arm on which the weight of the instrument driver carriage 1854 acts. In order to maintain stability and precise control, the movement of surgical tools 1856 on the base 1852 and the movement of the robotic arm 1936b must be slow. These components also require large electrical power draw affecting heat build-up and system durability. In order to account for the inherent lever arm-like action of drivers of the second type 1850, the robotic arm 1936b may have optimized construction to increase the stability of the arm 1936b for maintaining precise positions of the surgical tool 1856, the shaft 1858, and coupled end effector (not visible). For example, the linkages 1818 may be made of a more robust material, a stiffer material, or may have increased size dimensions compared to robotic arms 1936a. The actuators and motors operating each joint 1820 may also be larger and produce more torque than those actuators and motors used in the joints of robotic arms 1936a designed for use with instrument drivers of the first type 1840 and attached surgical tools 1300.

It is noted that in the distal position (FIGS. 19B and 19D), the instrument drivers 1840 and 1850 are most alike in terms of weight distribution. That is, the large mass of the instrument driver 1840 of the first type and instrument driver carriage 1854 of the instrument driver of the second type 1850, each housing the various motors to dive coupled surgical instrument functions, are at a minimal distance from the point of instrument driver rotation 1984 about the distal most joint 1820. Likewise, in the fully proximal positions illustrated in FIGS. 19A and 19C the instrument drivers 1840 and 1850 are the least alike in terms of weight distribution.

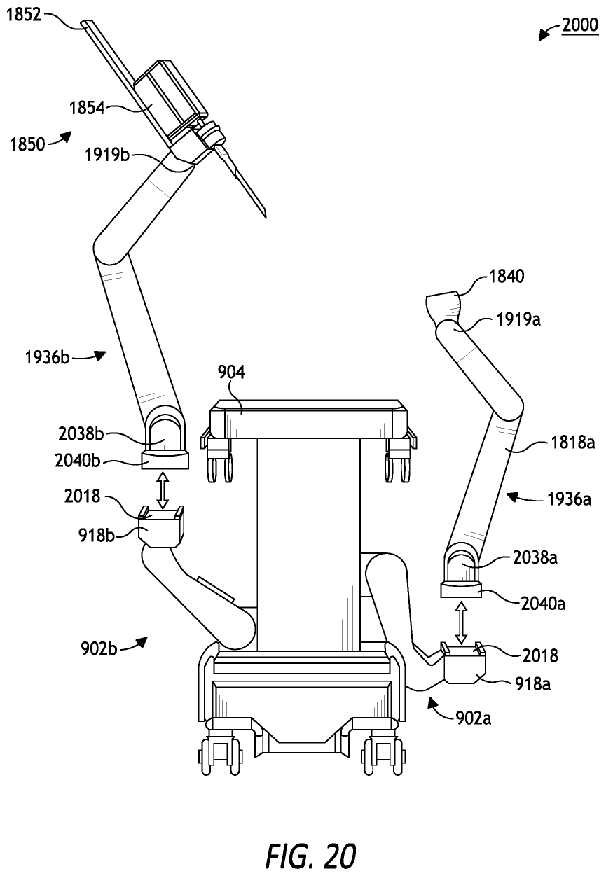

FIG. 20 illustrates an end view of an example robotic surgical system 2000, similar in some aspects to robotic systems 900 and 1800 of FIGS. 9 and 18, respectively. The robotic surgical system 2000 includes two adjustable arm supports 902a and 902b mounted on opposite sides of the table 904, each with rails 918a,b configured to removably receive robotic arms of varying types, e.g., robotic arms 1936a, 1936b, each described above with regard to FIGS. 19A-19D. The first robotic arm 1936a is of a first type and is removably attached to the first bar or rail 918a of the first adjustable arm support 902a. The removable attachment of the first arm 1936a to the first rail 918a is achieved via a modular base 2038a located at a proximal end of the base linkage 1818a. The modular base 2038a may include a modular interface 2040a configured to mechanically and electronically engage a complementary rail interface 2018. The modular interface 2040a and rail interface 2018 may provide one or more matable features configured to secure and/or electronically couple the robotic arm 1936a to the rail 918a. In some embodiments, the connection between the modular base 2038a and rail 918a is a fixed connection, i.e., the base 2038a does not move or pivot on the rail 918a. In other embodiments, the connection between the modular base 2038a and rail 918a allows for movement of the modular base 2038a with respect to the rail 918a, i.e., the base 2038a may rotate on the rail 918a and/or slide along a long axis of the rail 918a.

The robotic arm 1936a of the first type is generally characterized by the instrument driver 1840 attached at the distal joint 1919a of the robotic arm 1936a. As discussed above, the instrument driver 1840 is an instrument driver of the first type, and is stationary. Accordingly, the robotic arms of the first type 1936a may include shaft insertion through the instrument driver 1840. Since the mass of the instrument driver 1840 is substantially located at the distal joint 1919a, the structural components of the first arm 1936a (e.g., linkages, actuators, motors, etc.) may be optimized in terms of size, weight, and power draw to efficiently move, drive, and maintain the position of the instrument driver 1840 and attached surgical tool during a procedure. For example and without limitation, the size of the brakes located within joints 1820, configured to control movements of the connected linkages 1818, may be reduced, compared to those of robotic arms 1936b. As another illustrative example, less amperage may be required to hold a desired position of the arms 1936a with instrument driver 1840, therefore, the electrical power needed to maintain a position is reduced.

The second robotic arm 1936b may be of a second type. Similar to the robotic arm of the first type 1936a, the second robotic arm 1936b may be removably attached to the second bar or rail 918b. That is, the robotic arm of the second type 1936b includes a modular base 2038b with a modular interface 2040b for removably connecting, both mechanically and electronically, to the second rail 918b via a complementary rail interface 2018. The robotic arm 1936b of the second type is generally characterized by the instrument driver 1850 attached at the distal joint 1919b of the robotic arm 1936b. As discussed above, the instrument driver 1850 is an instrument driver of the second type and includes the instrument driver carriage 1854 that is mobile on the base 1852. Since a large mass of the instrument driver 1850 is mobile on the base 1852 and connected to the distal joint 1919b, the structural components of the second arm 1936b (e.g., linkages, actuators, motors, etc.) may be optimized in terms of size and weight to efficiently move, drive, and maintain the position of the mobile instrument driver carriage 1852 of the instrument driver 1850 of the second type and attached surgical tool during a procedure. In some embodiments, the second arm 1936b generates a significant amount heat due to the power requirements for moving the heavy stage-based instrument driver 1850 and connected tool. Thus, the components of the second arm 1936b may require heat to be dissipated, e.g., with the addition of cooling fans and/or cooling fins, or be configured to receive blown air.

In some embodiments, the modular interfaces 2040a,b may be compatible with the corresponding rail interfaces 2018, thus allowing for a direct connection between the two components. In other embodiments, one of or both of the modular interfaces 2040a,b may not be directly compatible with the corresponding rail interface 2018. In these cases, a modular adapter (not illustrated) having an interface compatible with both the rail interface 2018 and modular interface 2040a,b may facilitate mechanical and electrical connection between the rail 918a,b, and robotic arm 1936a, 1936b.

While the illustrated embodiment shows one robotic arm of the first type 1936a attached to one side of the table 904 and one robotic arm of the second type 1936b attached to the other side of the table 904, it is to be appreciated that each rail 918a, 918b may accommodate multiple robotic arms of any type. Furthermore, while two types of arms 1936a, 1936b are illustrated, other configurations of robotic arms in the prior art or future art may be removably attached to the surgical system 2000, without departing from the scope of the disclosure. In some embodiments, attachment of the arms 1936a, 1936b to the rails 918a,b may be facilitated by an adapter, as described above, or by replacing/interchanging one or more interfaces 2018, 2040a.b.

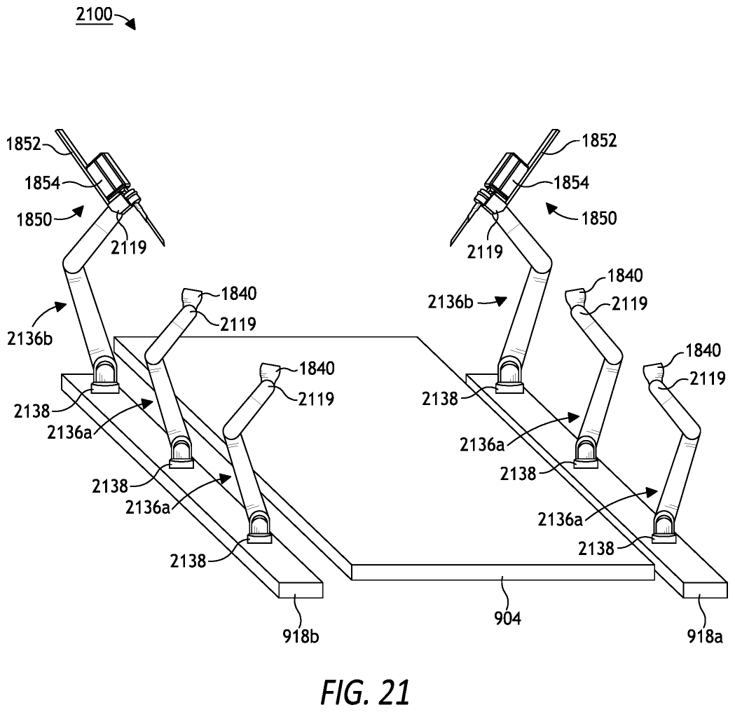

FIG. 21 illustrates a perspective view of another example robotic surgical system 2100, similar in some aspects to robotic systems 900, 1800, and 2000 of FIGS. 9, 18, and 20, respectively. The robotic surgical system 2100 includes two adjustable arm supports (not illustrated) mounted on opposite sides of the table 904 each configured to support the rails 918a,b, respectively. Each rail 918a,b is configured to receive at least one modular robotic arm 2136a and 2136b.

The surgical system 2100 includes one or more stationary instrument driver of the first type 1840, that drive shaft insertion through the instrument drivers. The surgical system 2100 also includes one or more instrument drivers of the second type 1850, having mobile instrument driver carriages 1854 on a coupled base 1852. The combination of instrument drivers of the first type 1840 and instrument drivers of the second type 1850 allows for the use of the simplest instrumentation possible for a particular procedure, e.g. a laparoscopic procedure, including but not limited to gastric bypass, lung cancer removal of a lobe, and lower anterior resection. That is, the instrument drivers of the first type 1840 may drive simple insertion shaft tools for grasping and dissecting which may be driven fast with great control while instrument drivers of the second type 1850 may driver higher power more complex instruments. For example, and without limitation, in an orthopedic procedure, instrument drivers of the second type 1850 may be control a tool for bone cutting which instrument drivers of the first type 1840 may control a tool for soft tissue dissection. In some embodiments, a distal most joint 2119 of each robotic arm of the surgical system 2100 is a modular joint, similar in many respects to the modular joint 1819 of FIG. 18. Modular distal joints 2119 are configured to receive any instrument driver, e.g., instrument drivers of the first type 1840 or instrument drivers of the second type 850, via modular interfaces described above.

In some embodiments, a base 2138 of each robotic arm 2136a,b of the surgical system 2100 may be a modular base for removably coupling a robotic arm 2136a,b, to one of the rails 918a,b, similar in many respects to the modular base 2038 of FIG. 20. The modular bases 2038 may include a modular interface 2040 (FIG. 20) configured to engage a complementary rail interface 2018 (FIG. 20). In yet other embodiments, the surgical system 2100 may have some distal joints 2119 that are modular and some proximal bases 2138 that are modular.

While illustrated as a bed-based robotic surgical system 1800, the modularity of the robotic arms and joints are applicable to other robotic surgical systems, for example and without limitation, cart based systems like those described with respect to FIGS. 1-3. That is, the robotic arms 104 of a cart-based system may include a modular joint 1819 configured to receive additional linkages 1818 and/or instrument drivers 1840, 1850. Additionally or alternatively, modularity of a robotic system can be achieved with a cart-based system that employs separate carts for separate robotic arms. In such embodiments, the cart-based system may include one or more first carts each having a first type of robotic arm (e.g., arm 1936a) and/or a first type of instrument driver (e.g., stationary instrument driver 1840), and one or more second carts each having a second type of robotic arm (e.g., arm 1936b) and/or a second type of instrument driver (e.g., mobile instrument driver 1850). Multiple such carts may be utilized and positioned in various locations with respect to a surgical bed to access surgical site(s) of the patient, wherein each arm/instrument driver selected may be appropriate for the type of actions performed with the respective arm/instrument driver. Additionally or alternatively, a combination cart and bed-based system may be utilized where the cart and bed-based portions employ different arm/instrument driver types. In such embodiments, the bed-based portion of the system may include one or more of a first type of robotic arm (e.g., arm 1936a) and/or a first type of instrument driver (e.g., stationary instrument driver 1840), and the cart-based portion may include one or more of a second type of robotic arm (e.g., arm 1936b) and/or a second type of instrument driver (e.g., mobile instrument driver 1850).

### 4. Implementing Systems and Terminology

Implementations disclosed herein provide systems, methods and apparatus for instruments and instrument drivers for use with robotic systems. It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The foregoing previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

As used herein, the terms “generally” and “substantially” are intended to encompass structural or numeral modification which do not significantly affect the purpose of the element or number modified by such term.

To aid the Patent Office and any readers of this application and any resulting patent in interpreting the claims appended herein, applicants do not intend any of the appended claims or claim elements to invoke 35 U.S.C. 112(f) unless the words “means for” or “step for” are explicitly used in the particular claim.

What is claimed is:

1. A robotic surgical system, comprising:
a stationary instrument driver comprising a first instrument driver body attached to a distal joint of a first robotic arm and operable to drive one or more functions of a first surgical tool; and
a mobile instrument driver comprising an instrument driver carriage translatable along a longitudinal base and operable to drive one or more functions of a second surgical tool, wherein the longitudinal base is attached to a distal joint of a second robotic arm.

2. The robotic surgical system according to claim 1, wherein the stationary instrument driver and the longitudinal base of the mobile instrument driver are pivotally coupled to the corresponding distal joints.

3. The robotic surgical system according to claim 1, wherein the stationary instrument driver is operable to translate a shaft of the first surgical tool through the stationary instrument driver.

4. The robotic surgical system according to claim 1, further comprising an instrument driver interface located on at least one distal joint, the instrument driver interface configured to receive the stationary instrument driver and the mobile instrument driver.

5. The robotic surgical system according to claim 1, wherein each robotic arm comprises at least two elongated linkages pivotally connected at a joint.

6. The robotic surgical system according to claim 1, wherein the first robotic arm has at least six degrees of freedom.

7. The robotic surgical system according to claim 1, further comprising a modular base, wherein a proximal end of each robotic arm and a coupled instrument driver are removably attachable to the surgical system at the modular base.

8. The robotic surgical system according to claim 1, further comprising a cart, wherein each robotic arm is removably attachable to an arm mount movably attached to the cart.

9. The robotic surgical system according to claim 1, further comprising:
a table; and
at least one rail located on a side of the table, wherein each robotic arm is removably attachable to the at least one rail.

10. A robotic surgical system comprising:
a first type robotic arm having a stationary instrument driver coupled to a distal end of each first type robotic arm;
a second type robotic arm having a mobile instrument driver including an instrument driver carriage capable of translation on an elongated base coupled the second distal end of the second type robotic arm; and
a modular base located on a proximal end of each of the first and second type robotic arms and operable to removably attach the first and second type robotic arms to the robotic surgical system.

11. The robotic surgical system according to claim 10, further comprising at least one elongated rail providing at least one rail interface operable to removably receive the modular base of each of the first and second type robotic arms.

12. The robotic surgical system according to claim 11, wherein at least one of the first and second type robotic arms mounted to the at least one rail is movable along a long axis of the at least one rail.

13. The robotic surgical system according to claim 10, wherein the stationary instrument driver operates to translate a shaft of a coupled surgical tool through the stationary instrument driver.

14. The robotic surgical system according to claim 10, further comprising a modular joint located on the distal end each of the first and second type robotic arms and operable to removably attach one of the stationary instrument driver and the mobile instrument driver.

15. A robotic surgical system comprising:
at least two robotic arms, each having a proximal end and a distal end;
one or more stationary instrument drivers configured to drive functions of a coupled first surgical tool; and
one or more mobile instrument drivers including an instrument driver carriage capable of translation on a longitudinal base,
wherein the one or more stationary instrument drivers and the one or more mobile instrument drivers are each removably attached to the robotic system at one of a modular joint located on the distal end of a robotic arm and a modular base located on the proximal end of a robotic arm.

16. The robotic surgical system according to claim 15, wherein the stationary instrument driver allows translation of a shaft of a coupled first surgical tool through the stationary instrument driver.

17. The robotic surgical system according to claim 15, further comprising a table and at least one rail located on a side of the table, wherein the at least two robotic arms attach to the at least one rail.

18. The robotic surgical system according to claim 15, further comprising a cart, wherein the at least two robotic arms removably attach to an arm mount movably attached to the cart.

19. The robotic surgical system according to claim 15, wherein each of at least two robotic arms comprise at least two elongated linkages pivotally connected at joints.

20. A method, comprising:
attaching a stationary instrument driver to a distal joint of a first robotic arm of a first type;
attaching a mobile instrument driver to a distal joint of a second robotic arm of a second type, the mobile instrument driver including an instrument driver carriage translatable along an elongated base;
driving one or more functions of a first surgical tool with the stationary instrument driver; and
driving one or more functions of a second surgical tool with the mobile instrument driver.
